SEQUENCE LISTING (58 sequences)

SEQ ID NO: 1 - DNA, 825 bp, Oryza sativa
atggctgctgctacttcgaccatgtccctgcttcctcccatcacccagcagcagcggtggcacgccgccgactccctcgtcgtcctcgcctcccgctgccacaactctcgccgccgccgccgctgccgctacgtcgtgccgagggcgaggctgttcgggccggcgatcttcgaggcgtcgaagctgaaggtgctgttcctgggggtggacgaggagaagcaccagcacccggggaagctgccgcggacgtacacgctgacgcacagcgacgtgacggcgaggctgacgctggcggtgtcgcacaccatcaaccgggcgcagctgcaggggtggtacaacaagctgcagcgggacgaggtggtggcggagtggaagaaggtgcagggccacatgtcgctgcacgtccactgccacatctccggcggccacgtcctcctcgacctcatcgccggcctccgctactacatcttccgcaaggagctccccgtggttctgaaggcgttcgtccacggcgacggcaacctgttcagccggcacccggagctggaggaggccacggtgtgggtctacttccactccaacctcccacgcttcaaccgcgtcgagtgctggggcccgctccgcgacgccggagcgccgcccgaggaagacgacgccgtcgccgccgcggcggccgaggaggcggcggcggagcagatgcccgcggccggcgagtggccgcggcggtgcccggggcagtgcgactgctgcttcccgccatacagcctcatcccctggccgcaccagcacgacgtcgccgccgccgacggccagccgcagcagtga

SEQ ID NO: 2 - DNA, 846 bp, Hordeum vulgare
atggccatcgccgctgccgctggcgcctccaccatgtccctgctccccatctcgcacctcaagcagctgcagctgcagcggcgcgcgcgccccgggcgggtgctcgtgctcggccgccggaggcgacacgtcgtgccgagggcgcggctgtttggtccggccatcttcgaggcgtccaagctcaaggtgctgttcgtgggggtggacgaggagaagcacccggggaagctgccccggacctacacgctcacccacagcgacgtgacggcgcggctgacgctggccgtgtcgcacaccatccacgccgcgcagctgcagggctggtacaaccgcctgcagcgggacgaggtggaggccgagtggaagaaggtgcagggcgccatgtcgctgcacgtccactgccacatctccggcggccacttcctgctcgacctcatcgcgccgctccgctactacatcttccgcaaggagctccccgtggttctgaaggcgttcgtgcacggcgacggcagcctgttcagccagcacccggagctggaggaggccacggtgtgggtctacttccactcgaacaaccccaacttcaaccgcgtcgagtgctggggcccgctcagcgacgccgccgcgccatacgatgacgaagccgccgtcgactccccagccgccgacgcagccatggcggccacggcggtgaacacggccgcggacgagcaggcgacgcgcgcgggccagtggccgcggcggagccccgggcagagcgactgctgcttcccgccggagtgcctcatcccctggccgcacgagcacgagatggccgccgacgccggccaggcgccgccgcagtga

SEQ ID NO: 3 - DNA, 798 bp, Triticum aestivum
atggccaccgcctccaccatgtccctgctccccatctcgcacctcaagcagctgcagcagcagcggcgcacgcggctcgccggcgcgggccccgggaaggtgctcgtgctcggccgccggaggcgacacgtcgtgccgagggcgcggctgttcggcccggccatcttcgaggcgtccaagctcaaggtgctgttcgtggggatggacgaggagaagcacccgggcaagctgccccggacctacacgctcacccacagcgacgtgacggcgcggctgacgctggcggtgtcgcacaccatccacgccgcgcagctgcagggctggtacaaccgcctgcagcgggacgaggtggtggccgaatggaagaaggtgcagggcgccatgtcgctgcacgtccactgccacatctccggcggccacttcctgctcgacctcatcgcgccgcttcgctactacatcttccgcaaggagctccccgtggttctgaaggcgttcgtgcacggcgacggcagcctgttcagccagcacccggagctggaggaggccacggtgtgggtctatttccactccaacaccccaaacttcaaccgcgttcagtgctggggcccgctcgcgaagccgcgggccctagacaacaagacgccgacgcggccgtgcccgcaaggcgacgccggggacaaaaaggcaatggatcgggcagcgccgcgggggtcccggggcatggaatgtttttcccgcccgaatcctatccctggcccaagaattcaaatgcccccaccccgccaggccccccaataa

SEQ ID NO: 4 - DNA, 795 bp, Triticum aestivum
atggccaccgcctccaccatgtccctgctccccatctcgcacctcaagcagatgcagcagcagcggcgcacgcggctcgccggcgcgctccccgggaaggtgctcgtgctcggccgccgcaggcgccacgtcgtgccgcgggcgcggctgtttggtccggccatcttcgaggcgtccaagctcaaggtgctgttcgtgggggtggatgaggagaagcacccgggcaagctgccgcggacctacacgctcacccacagcgacgtgacggcgcggctgacgctggcggtgtcgcacaccatccacgccgcgcagctgcagggctggtacaaccgcctgcagcgggacgaggtggtggccgagtggaagaaggtgcagggcgccatgtcgctgcacgtccactgccacatctccggcggccacttcctgctcgacctcatcgcgccgctccgctactacatcttccgcaaggagctccccgtggttctgaaggcgttcgtgcacggcgacggcagcctgttcagccagcacccggagctggaggaggccacggtgtgggtctactttcactccaacaaccccaacttcaaccgcgtcgagtgctggggcccgctcgcgatgccgcgcgccctagacgacgagacgccacgcgactcccaccggcgacgcaccgtgccactgcacgacgacagccgtcgcgcgggcagtgccccgggggccccggcattggatggtgttccgcaaaatgctatccctggcgcggacccaattgccgccaaccgccagggcccccaataa

SEQ ID NO: 5 - DNA, 846 bp, Zea mays
atggccgccgccgcttctaccatgtccctgctcccgatctcccagcccaggaagcagcagcagcaaggcgcgggcgccgtggtcgtgttccagcggcggccctgggacgcgcggcggaggcgatacgtcgtcccgacggcgaggctgttcgggccggcgatcttcgaggcgtccaagctgaaggtgctgttcctgggcgtggacgaggggagcagcaagcatctgcatgcgcaccacccggcgccggcgccgctgctgccgcggacgtacacgctgacgcacagcgacgtgacggccagcctgacgctcgccgtctcccacaccatcaaccgcgcgcagctgcagggctggtacaaccgcctgcagcgcgacgaggtggtggccgagtggaagaaggtgcgcggccggatgtcgctgcacgtgcactgccacatctccggcggacacttgctcctggacctcatcgccggcctccgctactacatcttccgcaaggagctccccgtggtgctcgaggcgttcgtgcacggcgacggcgacctgttcagccgtcacccggagctggaggaagccacggtgtgggtctacttccactccaacctggcccgcttcaaccgcgtcgagtgctggggtccgctccgcgacgccgccgcccccgcgcccgccgaggacgactccaccgcgccggccgccgcttccatcgccatggagggccagatgcccgtgggcgagtggccgcaccggtgtccccagcagtgcgactgctgcttcccgccgcacagcctcataccctggccgaacgagcaagacatggccgccgccgccggccaggtccgacagcagtag

SEQ ID NO: 6 - DNA, 825 bp, Zea mays
atggccgcagccaccgccgccgcttccaccatgtcgctgctcccgatctcccagctcaggcagcagcacggcgcgggcgccatgaggcggcggccctgggtcgcgcggcggaggcgatacgtcgttccgacggcgaggctgttcgggccggcgatcttcgaggcgtcgaagctgaaggtgctgttcctgggcgtggacgacgaggcgggcagcaagcagcacgggccgctgccgcggacgtacacgctgacgcacagcgacgtgacggccaggctgacgctcgccgtctcgcacaccatcaaccgcgcgcagctgcagggctggtacaaccgcctgcagcgcgacgaggtggtggccgagtggaagaaggtgcgcggccggatgtcgctgcacgtgcactgccacatctccggcggccacttcctgctcgacctcatcgcgggcctccgctacgtcattttccgcaaggagctccccgtggtgctcaaggcgttcgtgcacggcgacggcgacctgttcagccggcacccggagctggaggaggccacggtgtgggtctacttccactccaacctggctcgcttcaaccgcgtggagtgctggggtccgctccgcgacgccgccgcgcccgccgaggacgactccaccgcgccgcccgacgcctccaactccaaggaggccggccagatgatggccatgtgcgagtggccgcaccggtgtccccagcagtgcggctgctgcttcccgccgcacagcctcatcccctggccgaacgagcacgacatggccgccgcagatgcctccggctccgcccaacagcagtag

SEQ ID NO: 7 - DNA, 801 bp, Sorghum bicolor
atggccgcagccactgccgccgccgcttctaccatgtccctgcccccgatctcccagctcaggcagcagcagcacggcgcgggcgccgtggtcgtgttccggcggcgggcccgggacgcgcggcggaggcgatacgtcgtgccgacggcgaggctgttcgggccggcgatcttcgaggcgtccaagctgaaggtgctgttcctgggcgtggacgaggagagcaacaacaagcacgggcacccgacgacgccgtcgccgacttccccgccgctgccgctactgccgcggacgtacacgctgacgcacagcgacgtgacggccagcctgacgctggccgtgtcccacaccatcaaccgcgcgcagctgcaagggtggtacaaccgcctgcagcgggacgaggtggtggcggagtggaagaaggtgcgcgggcggatgtcgctgcacgtgctcaaggctttcgtgcacggcgacggcgacctgttcagccggcacccggagctggaggatgccccggtgtgggtctacttccactccaacctgacccgcttcaaccgcgtcgagtgctggggtccgctgcgcgacgccgccgcgccgccggccgaggacgactccaccgcgccggccgccgcctccaacaaggatgggcagatgccgcccgtgggcgagtggccgtaccggtgtccccagcagtgcgactgctgcttcccgccgcacagcctcatcccctggccgaacgagcgcgacatggcggccgccgccgccgatgcctcctccgccgccggccaggcccaacagcagtag

SEQ ID NO: 8 - DNA, 786 bp, Glycine max
atgtgtactctcacaactgttcctgtgctcccttctaagcttaacaagccttcgctttctccgcaccacaattctctttttccctactgtggaagacgggtcgggaagaagaacaaagcaatggttcctgttgcaaggttgttcgggccagccatatttgaagcctcaaaactgaaggttttgttcttaggagtggacgaaaataagcacccaggaaatctcccaaggacttatacgctaacccatagtgatataaccgctaagctcaccttggcaatctctcaaaccataaataattctcagctgcaggggtggtacaacagatttcaaagggacgaagtggtggcacagtggaaaaaggtgaagggaaggatgtctctgcacgttcactgccacattagtggaggtcattttctcttggatatattagcaaggttaagatacttcatcttctgcaaggagctaccagtggtgttgaaggccgtcgttcacggcgatgaaaacctattcaacagctacccagaattgcaagatgccttggtttgggtctactttcactcaaacattccagaattcaacaaggtggaatgttggggcccactgaaggaagcgtcagcacccacaggtggggtccaggaggaggggttggcaattccacagccatgccaagaagaatgccaatgttgctttccaccgcttacgttgagccctattcagtggtctaaacaagttcccagccgccattacgaaccttgtgatgggattgggacccaacaaaatctataa

SEQ ID NO: 9 - DNA, 816 bp, Glycine max
atgggtactctaacaactgttcctgtgctcccttctaagcttaacaagccttcgctttctccgcgtcacaattctctttttccctactacggaagacgcgtcgggaagaagaacaaagcaatggttcctgttgctaggttgttcgggccagccatatttgaagcctcaaagcttaaggttttattcttaggagtggacgaaaataaacacccaggaaatctcccaaggacttatactctaacccatagtgatataaccgctaagctcaccttggcaatctctcaaaccataaataattctcagttacaggggtggtacaacagattgcaaagggacgaagtggtggcacagtggaagaaggtgaagggaaagatgtctctgcacgtacactgccacatcagtggtggtcattttctcttagatatattagcaaggttacgatacttcatcttctgcaaggagctaccagtggtgttgaaggcggtggttcacggcgacgaaaacctattcaacaactacccagaattgcaagatgccttggtttgggtttactttcactcaaacattccagaattcaacaaggtggaatgttggggcccactgaaggaagcgtcagcaccaataggtggggccaaggaagagagtgagcaagaaactcttctaagtaaggagggcttggcaattccacagccatgccaagaggaatgcgaatgttgctttccaccgctgacgttaagcccaattcagtggtctcaacaagttcccagccaccattacgaaccttgtgatgggattgagacccaacaaagtctataa

SEQ ID NO: 10 - DNA, 825 bp, Vitis vinifera
atggctactttgactgctgctcttgtgcttccgtctgagctcaaaccttctttctctcaacaccaaagttctctcttcgtttgtcgaagaagaccaaagaagagtaaccctgcttttcctgccgcaaggctgtttggtcctgcaattttcgaagcttcaaagcttaaggttctgtttttgggagtggatgagaagaagcacccagggaagcttcctagaacttacacgcttacgcatagtgacataacatctaaactcactctggctatatctcaaactataaacaactctcagttgcaggggtggtccaacagattacaaagagatgaggtggtggcacaatggaagaaagtgaaagaccagatgtctctgcatgtgcactgccacataagtggaggccatttccttctagatttgtgcgctaaacttagatacttcatcttctgcaaagagcttccagtggttttgaaggcttttgttcatggagatggcaacctgctcaacaattacccagaattacaggaagctttggtttgggtttactttcactcgaacctcccagaattcaatagagtagaatgctggggggcgctcaataatgcagcggcgcctcctcctcctgccgccggtggtggcggtggtagggtggaggcacaccaggacatgaggcaggtggaaccatcaagcaaatgggagaggccggaagagccatgcatggagaactgtacatgttgcttcccaccaatgagcctcatcccatggtcacaagatctcgcccatgaaaatattcatgatacccaaaagggattacagcagcaaacctga

SEQ ID NO: 11 - DNA, 843 bp, Lactuca sativa
atggcttctctgatccttcccacaaagcaaaaccctccatcgtcttcgtttctgcatcaaaatcatcaaaacaatccgttttttactaacaaaagacgaaagctcaagaggaatcaagccctagttcccgttgcaagattatttgggccttcgatctttgaagcttcaaagttgaaggttttgtttctaggagttgacgagaagaagcatcctggaaaacttccaagaacatatacacttacacatagtgatatcacgtctaaattgactctggcaatctctcaaactatcaataattctcagttgcagggttggtataaccaattatacagagatgaagtggtagcagagtggagaaaagtgaaagggaatatgtctcttcatgttcattgtcacataagtcgtggccattttcttcttgatttgtgtgctcgactcaggttcttcatcttcaccaaagaactccctctggtgttgaaggcatttgctcatggagatgggaatttgctaaacagctacccggagttgcaggaagcttcggtttgggtttactttcactcaaacattcaagaattcaatagggttgaatgttgggggccactcagagaagcagtgggacccttatccaccaccacttcatcatcatcatcatcatcattatctgaatccaccattgctgaagctggagaaggatcaaacaattgggagatcccaaagccatgtctagaagcatgtgcatgttgctttccaccgatgagttcaatcccatggtcacatgatcttgtgaagaatcaagacgatgatgatggtgccacccaccaagggttgcaacaaaaagcttga

SEQ ID NO: 12 - DNA, 873 bp, Pinus taeda
atggcggtggcaagaatctctgcaggaaaaacacagcactgctactccttctccccatctgatgtacggatttcgtctgcaccacagaattcacagtctcagttcaaaaggaaatcgaagataaagctttcctccaggtttctggccagcgagagcagctggaatggcctggtcgcgcatcagttacagtgcaataacagacatcgaactaatagcagcttcccccgatccaccagtcgtgtggtggcgagattgtttgggcctgcaatcttccaggcatcgaagctcaaggttctatttcttggaacacatgaagagaaacatcctgcgcatcttcccaggacttatacgctcacacacagcgacatcacggccaaattaacgctggctttttctcaaacaatcaataaagatcagggatggtataacaggttacagagagacgaagttcttgcgcagtggaagaaatctcagggcaaaatgtctctgcacgttcactgtcacatcagcggaggtcactggctcctggacgccattgctagacttagattttacatcttccgcaaggaactgccggtggtgctggaggcgttcagacatggggaccgggctctgcttgagaagcacccagagctggagaccgcactggtttgggtgtattttcattccaatgtcaaagaattcaaacgtgtggaatgttgggggtctttggctgaagcatgcaagggtgcacctagcaatttgaacaaggaattggacgagctcgatggtggaaaattggagatgcctagtcattgcgcagaaccatgtagttgttgctttcctccctttagtgttcttctacgaccagaagatgttgaacaatttagctaa

SEQ ID NO: 13 - DNA, 816 bp, Citrus sinensis
atggctagtttggttgctgctcttgggcttccctcaaagctcaaagcttccccctatgagcagcaaaacgcactctttgtttctagaagaagatccaagaaaaagaaccaatcttttgctcctgtggcaagattattcggaccagccatttttgaagcttcaaagctgaaggtattgtttttgggggtggatgaagagaagcatccagggaagctgccaaggacttatacacttacccatagtgatataacctctaagcttactttagctatttctcaaaccataaataattctcagctgcagggatggtacaacaggttgcaaagggatgaggttgtggcagagtggaagaaggtaaagggaaagatgtctcttcatgttcactgtcacataagtggaggccatttcttattagacatttgtgctagacttagattcttcatcttctccaaggaactccccgtggttctgaaggcatttgttcatggagatggcaatttgttaaacaatcacccggaattacaggaggctttggtttgggtctattttcattccaatattcctgaattcaataaagtcgaatgctggggtccactcaaagaggcagttgccggatcgagtgaagctggcgggacccgccacgagattaggcaagaaacttcaataagcaactgggaattaccagaaccctgccaggaaacgtgcaactgttgctttcctccaatgagcttgatcccgtggtcagagaagcttccccttcaaaccgaaaatcgtgggacccagggccaagaaagcttacagcaacaaacccgatga

SEQ ID NO: 14 - DNA, 792 bp, Medicago truncatula
atgggtactctaaccaccgctcctcctcctatgctcacttctaagttcaaaccttctttttcacctcaacataaacctctttttccaaatagaagacggttatggaagaagaaccaatcaattgttcctgttgctaggttatttggaccggctatatttgaagcatcaaaattgaaggttttgttcttaggaattgatgaagacaaacatccaggaaatcttccaaggacttatacgttaacacatagtgatgtaacctcaaaactcactttggcaatttctcaaaccattaataactctcagttgcagggatggtataatagattgcaaagggatgaagttgtggcgcagtggaagaaggtgaagggaaagatgtctctccatgttcattgtcatattagtggtggccattttttgttagatatatttgctagactaagatatttcatcttctgcaaagagttacccgtggtattgaaggcttttgtacacggtgacggcaatttattcaacaactatccggaattacaggaagcattggtttgggtatattttcattcaaagattccagaattcaacaaggtagaatgttggggtccactaaaggaggcttcacaacctactagtgggacccaaagggaccaccaaaatttgaccctacctgagccatgtcaagaaacttgcgagtgctgctttccaccgttgaagttgagcccaatgccgtgctctaatgaggttcacaatgatacttatgaacctattgatggaattgaaactcaacaatcactgtaa

SEQ ID NO: 15 - DNA, 819 bp, Solanum tuberosum
atgggaactttgactgcttctctagtggttccatctaagctcaacaatgaaaaacagagctctatttttgtacacaaaactagaagaaaatccaagaagaatcaatccatagtacctgtggcaaggttatttgggccagctatatttgaagcttcaaagttgaaggtactttttttgggagttgatgaggaaaagcatccaggaaagttgccaagaacatatacactgactcatagtgatattacttctaaacttactttggctatctctcaaaccatcaataactctcagttgcaaggttggtataatagacttcaaagagatgaagttgttgcagaatggaagaaagttaaagggaagatgtcacttcatgtccattgccacataagtggaggccattttatgttagacttatttgctagactcagaaactatatcttctgcaaagaactccctgtggttctgaaggcttttgttcatggagatgagaatttattaaagaataatccagagttacaagaagctttagtttgggtatattttcattcaaacattcaagaattcaacaaagtagaatgttggggtccactcaaagatgcaacctccccctcatcttcttctagtggggtaggtggggtgaagagtacaagttttacaagcaatagtaacaacaagtgggagttaccaaaaccttgtgaagaggcttgtgcatgttgctttcccccaatgagtgttatgccttggccttcttcaaatcttgatgggataggtgaggaaaatgggaccatccaacaaggcttgcaagagcagcaaagttga

SEQ ID NO: 16 - DNA, 810 bp, Populus tremula x Populus tremuloides
atgggctctctggcaattgctccctttcttccttcaaagctaagaccctctatacttgatcaaaatagctctctctttccttcaaagaaaaaactcaagaggaagaaccaatctatcagtcctgtggcaaggttatttgggccatctatttttgaggcatcaaaactgaaggtgttgtttttaggggttgatgagaagaaacatccagggaatctgccaaggacttatacactaacacatagtgatattacagctaaacttactttagccatctcacaaaccatcaacaattctcagttgcagggatggtccaacaaattgtacagagatgaagtggtggcagagtggaagaaagtaaagggaaagatgtctctccatgttcactgccatataagtggaggccattttctcctagatttatgttgtagacttagatatttcatcttccgcaaagaacttcctgtggtattgaaggccttctttcatggagatgggaatttgtttagcagctatcctgaattgcaggaggctttagtttgggtttactttcattccaacattccagaattcaacaaggtagagtgctggggtccactcaagcatgccgcagcaccttatactgctgcatctggcggggcccctgagaacaaggagcaagcaaccgactggaacttgcctgagccatgccaagagaactgtcagtgttgctttccaccaatgagcttgatcccatggtccgaaatggttccccaagagaacaagaataatccaagcacccagcagacctttcaacaagctcaacaaccctaa

SEQ ID NO: 17 - DNA, 813 bp, Populus tremula x Populus tremuloides
atgggttctttggcagttgctccctttcttccctcaaagccaagaccctctctctttgatcaacacagctccctcttttctccaagtacaaagctcaagaggaagaaccaatctatcagccctgtggcaaggttatttgggccatctatttttgaggcatcaaagctgaaggtgctgttcttaggggttgatgagaaggagcatccagggaatctgccaaggacttatactctaacacacagtgatatgacagctaagcttactttagccatctcacagaccataaacaattctcagttgcagggatggtccaacaaattgtaccgagatgaagtggtggcagagtggaagaaagtaaagggaaagatgtctcttcatgttcattgccatataagtggaggccattttcttttagattggtgctgcagactcagatatttcatcttccgcagagaactccctgtggtattgaaggccttttttcatggcgatgggagcttgttgagcaactatcctgaattacaggagggtttagtttgggtttactttcattcaaacattccggaattcagcaaggtcgagtgctggggtccactcaaggatgctgctgcgccttctacttctgaaactggtgggtccaatgagaccgaggagctagcaaaccaatcaagcaactgggacttgcccgagccatgccaagaggagaattgtagctgttgctttccaccaatgagcttgatcccatggtctaaaatggttccgttggaggacaaaaataatccaagcaccccacagaaccttcaacagccctaa

SEQ ID NO: 18 - DNA, 861 bp, Mesembryanthemum crystallinum
atgggcactttgactgcctctatgttgctcccatcaaagctcaaaccttcagtctttgaagatcaatcctctgtttattttaaaagatcatgcagaggacttcccaagctcaacaaggccaaatctttttcacctgtgatgagattgtttgggccagcaatatttgaagcatcaaagttgaaggtgttgttcttgggagtggataaagagaagcacccagggaagttgcctagaacttatactcttactcatagtgatatcacttccaagctcactttggccatctctcaaactattaacaattcccagttacaagggtggtacaaccaactacagagagatgaagtggtggcagaatggaagaaagtgaaagggaagatgtcactccatgttcattgtcacataagtggtggccatatcctcttagacttatttgctaagcttagattctacatcttttgcaaggaactccctgtggtattgaaggcatttgtgcatggggatgagaatttgttcaacaactacccagaactacaagaggcaatggtgtgggtatacttccattcaaaccttgaagaattcaacaaaatcgagtgctggggcccgctcaaggatgccgtggcacgcaactcgaagaaaaacaagaacaagaacaagatagatttcaagttaagtttcaaagaagaggatgattcaccagataacgagttggagataccagagacttgcaaggaaccctgtacctgttgctttcctcccactagtgtcatcccttggtctcattcagcattgtcacagggtgatgatcttcatctctctggtgggacccaccaaggcttggagcagcagcagcaaacttga

SEQ ID NO: 19 - DNA, 807 bp, Arabidopsis thaliana
atgtgtagtttgtcggcgattatgttgttaccaacgaagctgaaaccagcttattcagacaaacggagtaacagtagcagcagcagctcactcttcttcaacaatagaagatccaagaagaagaaccaatcgattgttcccgttgcaaggttgtttggaccggcgattttcgaatcatccaaattgaaagtactcttcttaggggttgatgagaagaagcatccttcaacgctccctaggacttacacactcactcacagtgacattacagctaaactaaccttagctatttctcaatccataaacaactctcagttgcaaggatgggcaaataggctataccgggatgaagttgtggcagaatggaagaaagtgaaagggaaaatgtcgcttcacgttcattgtcacataagcggtggccatttccttttagatctctttgcaaagtttcgatatttcatcttttgcaaagaactacctgtggtgttgaaggcttttgtgcatggagatgggaacttgttgaacaactatcctgagctacaagaagctcttgtttgggtctatttccattctaatgtcaatgagttcaacaaagtcgagtgttggggtccgctttgggaagctgtttcgcctgatggtcacaagactgagactcttcccgaggctcggtgtgcggacgagtgtagttgttgttttccaaccgttagctcgattccatggtctcatagtcttagtaatgaaggtgtaaatggttactctgggactcagactgagggaattgctactccaaatccggagaaactctag

SEQ ID NO: 20 - DNA, 816 bp, Arabidopsis thaliana
atgtgtagtttggctacaaatctgttactaccatcgaagatgaaaccagtttttccagagaaactgagcactagctcactctgtgtcaccactagaagatctaagatgaagaaccgatctattgttcctgttgcaagattgtttggaccggcgatttttgaagcctccaaattgaaagtgttattcttaggagttgatgagaagaagcatccagcaaaacttccaagaacttacactcttactcacagtgacataaccgctaaattaactttagctatatctcaatccattaataactctcagttgcaaggatgggcaaataaattgttccgggacgaagtagtgggcgagtggaagaaagtgaaaggtaaaatgtcgcttcatgttcattgccacattagcggaggccacttcttcttgaatctcatcgcgaagcttcggtactacatcttttgcaaagaattacctgtggtactggaagcttttgcccatggagatgagtatttgttaaataatcaccccgagctacaagaatctcctgtttgggtttatttccattccaacatcccggagtacaacaaggtcgaatgttggggaccgctttgggaggccatgtcgcagcaccagcacgacggaaggacccacaagaagagtgaaactctaccggagctaccttgtcctgatgagtgcaagtgttgctttccgacggttagcacgattccgtggtctcatcgtcattatcaacataccgcagcggatgagaatgttgcggatggcctgttggaaatacctaaccctgggaaatcaaagggatag

SEQ ID NO: 21 - DNA, 662 bp, Lycopersicon esculentum
atgggaactttgactacttctctagtggttccatctaagctcaacaatgaacaacagagctctatttttatacacaaaactagaaggaaatgcaagaagaatcaatccatagtacctgtggcaaggttatttggaccagctatatttgaagcttcaaaattgaaggtactttttttgggagttgatgaagaaaagcatccaggaaagttgccaagaacatatacactgactcatagtgatattacttctaaacttactttggctatctcccaaaccatcaataattctcagttgcaaggttggtataacagacttcaaagagatgaagttgttgcagagtggaagaaagtaaaagggaagatgtcacttcatgtccattgccacattagtggaggccattttatgttagacttatttgctagactcagaaactacatcttctgcaaagaactccctgtggttctcaaggcttttgttcatggagatgagaatttactaaggaattatccagagttacaagaagctttagtttgggtatattttcattcaaacattcaagaattcaacaaagtagaatgttggggtccactcagagatgcaacttccccctcatcttcttctggtggggtaggtggggtgaagagtacaagttttacaagcca

SEQ ID NO: 22 - DNA, 334 bp, Beta vulgaris
cccggaattacaagaagcttcagtatgggtatacttccattcaagcattcctgaatttaacaaagtagagtgctggggcccattgaccgacgccgtggatccgccgtcgaaaaataagaagaggatgatgatgataaatgatgagcaggataaagaagaagaagaagaagcaagtagctcaaaatgggagatgttagttccttgcacgaaaccatgtagatgttgctttccacctacaagtttgattccttggactccttcactatcacaagaacagcaacaagagcaacaacttcctggagacgtttcgatcccgccacctgggactcgctag

SEQ ID NO: 23 - DNA, 564 bp, Zosterops japonica
acgtacacgcttactcacagcgacgtcacggccaagctcacgctggcggtctcccacaccatccacgccgcgcagctgcaggggtggtacaaccgcctgcagcgggacgaggtggtggccgagtggaggaaggtgcgcgggaacatgtcgctgcacgtccactgccacatctccggcggacacttcctccgcgacctcatcgcgccgctccgctactacatcttccgcaaggagctccccgtggttctcaaggcgttcgtgcacggcgacggcagcctgttcagcagccacccggagttggaggaggccacggtgtgggtctacttccactccaacctgccccgcttcaaccgcgtcgagtgctggggtcctctctgcgacgccgccgcgcccgtcgaggaggaggggcagcagaatgacgatcggttgcccgcgggcgagtggccgcggcggtgcccccagcagtgcgagtgctgcttcccgccgcacagtctcatcccctggcccaacgagcacgacatggctcccaccgacgcccccgccgctggccagacgcagcagtga

SEQ ID NO: 24 - DNA, 284 bp, Lotus corniculatus
actacccagaattgcaggatgcattggtttgggtatactttcactcaaagattccagagttcaacaaggtacagtgttggggaccactgaaggaggcggctgcaccgtcaggtgggtccccggagaaagaaggtgaaggggtgaagatgccggatccgtgtccagaagaatgtgagtgttgctttcctcctccaccggcattggatccaatcccatggtctgaagaagttccctctccccattatgaagcttttgatggggttgggacccgaccaaacttgtag

SEQ ID NO: 25 - DNA, 326 bp, Lotus corniculatus
tagatctatgtgctaagctaagatacttcatcttctgcaaagagcttccagtggtattgaaggccttcattcacggcgatgaaaatttgttcaacaactacccggagttggaggaatcattggtttgggtttactttcactcaaacatctcagaattcaacaaggtggagtgttggggtccacttaaggatgcttgtgcaacatcaattgggtcctactcctatgacaagggtatgcctcaaactcagccatgccaacaaaactgcgagtgttgctttacaccgatgagctcaagtgattggattggaacccaacaaaaattgtga

SEQ ID NO: 26 - DNA, 415 bp, Saccharum officinarum
cacgaggctcgacctcatcgccggcctccgctactacatcttccgcaaggagctccccgtggtgctcaaggcgttcgtgcacggcgacggcgacctgttcagccggcacccggagctggaggatgccacggtgtgggtctacttccactccaacctgacccgcttcaaccgcgtcgagtgctggggtccgctccgcgacgccgccgcgccgccggccgaggaagactccaccgcgccggccgcctccaactccaaggaggggcagatgccgcccgtgggcgagtggccgtaccggtgtccccagcagtgcgactgctgcttcccgccccacagcctcatcccctggccgaacgagcacgacatggctgccgccgccgccgatgccaccgccgctggccaggcccaacagcagtag

SEQ ID NO: 27 - DNA, 481 bp, Picea abies
aatcaataaagatcagttgcagggatggtataacaggttacagagagacgaagtgattgcccagtggaagaaatctcagggcaaaatgtctctgcacgttcactgtcatatcagcggaggtcattggcttctggacgccatcgcgagacttagattttacatcttccgcaaggaactgccggtggtgctggaggcgttcaggcatggagatcgggctctgcttgacaagcacccagagctagagaccgctctggtttgggtgtatttccactccaatgtcagagagttcaaacgcgtggagtgttggggttctttggctgaggcatgcaagggtgcccctagcaatttggagaaggaattggacgaggagtttaatggtgaaaaattggagatgcctagtcattgctcagaaccatgcaattgttgctttcctccatttagcgtccttctacgaccagaagatgctgaacaatttatttaa

SEQ ID NO: 28 - DNA, 632 bp, Brassica napus
atgtgtagtttggcaacaaatctcttactcccatcgacgatgaaaccagcttttacagagaaacagaacactaactcactctttcttacaaataaaagatccttgatgcagaacagatctactgttcctgttcctgttgcaagattgttagaaccggcgatttttgaagcctccaaattgaaagtatcgttcttaggagttgatgagaagaagcatccatcaaagctcccaagaacttacactcttactcacagtgacataacagctaagttaactttagctatctcccaatctatcaataattctcagttgcagggatgggctaatagattatttcgggacgaagtagtggccgagtggaagaaagtgaagggtaaaatgtcccttcacgttcattgccacattagcggaggccacttccttttggatctcatagcgaagcttcggtactacatattttgcaaggaattaccggtggtattgaaagcttttgttcatggggatgggaacttgttgaatagttaccctgagctacaagaatctcctgtttgggtttattccattcaaacatccccgagtacaataaggttgaatgttgggggccgctttgggaggccacgcagcacaaacac

SEQ ID NO: 29 - DNA, 291 bp, Brassica napus
atgtgtagtttgtcagcgaacatgttgttaccgacaaagctgaaaccagcttattcagacaaacggggtaatagtacgaactcacttcttgtctccaatacaagatccaagaggaagaaccaatccgttgttcctatggcaagattgtttggaccggcgattttcgaatcatccaagttgaaagtattgtttctaggtgttgatgacaagaagcatccaccaacgcttccaaggacttacactctcactcacagtgacattacagctaagctaactttagctatttctcac

SEQ ID NO: 30 - protein, 274 aa, Oryza sativa
MAAATSTMSLLPPITQQQRWHAADSLVVLASRCHNSRRRRRCRYVVPRARLFGPAIFEASKLKVLFLGVDEEKHQHPGKLPRTYTLTHSDVTARLTLAVSHTINRAQLQGWYNKLQRDEVVAEWKKVQGHMSLHVHCHISGGHVLLDLIAGLRYYIFRKELPVVLKAFVHGDGNLFSRHPELEEATVWVYFHSNLPRFNRVECWGPLRDAGAPPEEDDAVAAAAAEEAAAEQMPAAGEWPRRCPGQCDCCFPPYSLIPWPHQHDVAAADGQPQQ

SEQ ID NO: 31 - protein, 281 aa, Hordeum vulgare
MAIAAAAGASTMSLLPISHLKQLQLQRRARPGRVLVLGRRRRHVVPRARLFGPAIFEASKLKVLFVGVDEEKHPGKLPRTYTLTHSDVTARLTLAVSHTIHAAQLQGWYNRLQRDEVVAEWKKVQGAMSLHVHCHISGGHFLLDLIAPLRYYIFRKELSVVLKAFVHGDGSLFSQHPELEEATVWVYFHSNNPNFNRVECWGPLSDAAAPYDDEAAVDSPAADAAMAATAVNTAADEQATRAGQWPRRCPGQCDCCFPPECLIPWPHEHEMAADAGQAPPQ

SEQ ID NO: 32 - protein, 266 aa, Triticum aestivum
MATASTMSLLPISHLKQMQQQRRTRLAGALPGKVLVLGRRRRHVVPRARLFGPAIFEASKLKVLFVGVDEEKHPGKLPRTYTLTHSDVTARLTLAVSHTIHAAQLQGWYNRLQRDEVVAEWKKVQGAMSLHVHCHISGGHFLLDLIAPLRYYIFRKELPVVLKAFVHGDGSLFSQHPELEEATVWVYFHSNNPNFNRVECWGPLREAAAPYDNKTPTRPCPQGDAGDKKAMDRAAPRGSRGMECFSRPNPIPGPRIQMPPPRQAPQ

SEQ ID NO: 33 - protein, 264 aa, Triticum aestivum
MATASTMSLLPISHLKQMQQQRRTRLAGALPGKVLVLGRRRRHVVPRARLFGPAIFEASKLKVLFVGVDEEKHPGKLPRTYTLTHSDVTARLTLAVSHTIHAAQLQGWYNRLQRDEVVAEWKKVQGAMSLHVHCHISGGHFLLDLIAPLRYYIFRKELPVVLKAFVHGDGSLFSQHPELEEATVWVYFHSNNPNFNRVECWGPLAMPRALDDETPRDSHRRRTVPLHDDSRRAGSAPGAPALDGVPQNAIPGADPIAANRQGPQ

SEQ ID NO: 34 - protein, 281 aa, Zea mays
MAAAASTMSLLPISQPRKQQQQGAGAVVVFQRRPWDARRRRYVVPTARLFGPAIFEASKLKVLFLGVDEGSSKHLHAHHPAPAPLLPRTYTLTHSDVTASLTLAVSHTINRAQLQGWYNRLQRDEVVAEWKKVRGRMSLHVHCHISGGHLLLDLIAGLRYYIFRKELPVVLEAFVHGDGDLFSRHPELEEATVWVYFHSNLARFNRVECWGPLRDAAAPAPAEDDSTAPAAASIAMEGQMPVGEWPHRCPQQCDCCFPPHSLIPWPNEQDMAAAAGQVRQQ

SEQ ID NO: 35 - protein, 274 aa, Zea mays
MAAATAAASTMSLLPISQLRQQHGAGAMRRRPWVARRRRYVVPTARLFGPAIFEASKLKVLFLGVDDEAGSKQHGPLPRTYTLTHSDVTARLTLAVSHTINRAQLQGWYNRLQRDEVVAEWKKVRGRMSLHVHCHISGGHFLLDLIAGLRYVIFRKELPVVLKAFVHGDGDLFSRHPELEEATVWVYFHSNLARFNRVECWGPLRDAAAPAEDDSTAPPDASNSKEAGQMMAMCEWPHRCPQQCGCCFPPHSLIPWPNEHDMAAADASGSAQQQ

SEQ ID NO: 36 - protein, 266 aa, Sorghum bicolor
MAAATAAAASTMSLPPISQLRQQQHGAGAVVVFRRRARDARRRRYVVPTARLFGPAIFEASKLKVLFLGVDEESNNKHGHPTTPSPTSPPLPLLPRTYTLTHSDVTASLTLAVSHTINRAQLQGWYNRLQRDEVVAEWKKVRGRMSLHVLKAFVHGDGDLFSRHPELEDAPVWVYFHSNLTRFNRVECWGPLRDAAAPPAEDDSTAPAAASNKDGQMPPVGEWPYRCPQQCDCCFPPHSLIPWPNERDMAAAAADASSAAGQAQQQ

SEQ ID NO: 37 - protein, 261 aa, Glycine max
MCTLTTVPVLPSKLNKPSLSPHHNSLFPYCGRRVGKKNKAMVPVARLFGPAIFEASKLKVLFLGVDENKHPGNLPRTYTLTHSDITAKLTLAISQTINNSQLQGWYNRFQRDEVVAQWKKVKGRMSLHVHCHISGGHFLLDILARLRYFIFCKELPVVLKAVVHGDENLFNSYPELQDALVWVYFHSNIPEFNKVECWGPLKEASAPTGGVQEEGLAIPQPCQEECQCCFPPLTLSPIQWSKQVPSRHYEPCDGIGTQQNL

SEQ ID NO: 38 - protein, 271 aa, Glycine max
MGTLTTVPVLPSKLNKPSLSPRHNSLFPYYGRRVGKKNKAMVPVARLFGPAIFEASKLKVLFLGVDENKHPGNLPRTYTLTHSDITAKLTLAISQTINNSQLQGWYNRLQRDEVVAQWKKVKGKMSLHVHCHISGGHFLLDILARLRYFIFCRELPVVLKAVVHGDENLFNNYPELQDALVWVYFHSNIPEFNKVECWGPLKEASAPIGGAKEESEQETLLSKEGLAIPQPCQEECECCFPPLTLSPIQWSQQVPSHHYEPCDGIETQQSL

SEQ ID NO: 39 - protein, 274 aa, Vitis vinifera
MATLTAALVLPSELKPSFSQHQSSLFVCRRRPKKSNPAFPAARLFGPAIFEASKLKVLFLGVDEKKHPGKLPRTYTLTHSDITSKLTLAISQTINNSQLQGWSNRLQRDEVVAQWKKVKDQMSLHVHCHISGGHFLLDLCAKLRYFIFCKELPVVLKAFVHGDGNLLNNYPELQEALVWVYFHSNLPEFNRVECWGALNNAAAPPPPAAGGGGGRVEAHQDMRQVEPSSKWERPEEPCMENCTCCFPPMSLIPWSQDLAHENIHDTQKGLQQQT

SEQ ID NO: 40 - protein, 280 aa, Lactuca sativa
MASLILPTKQNPPSSSFLHQNHQNNPFFTNKRRKLKRNQALVPVARLFGPSIFEASKLKVLFLGVDEKKHPGKLPRTYTLTHSDITSKLTLAISQTINNSQLQGWYNQLYRDEVVAEWRKVKGNMSLHVHCHISRGHFLLDLCARLRFFIFTKELPLVLKAFAHGDGNLLNSYPELQEASVWVYFHSNIQEFNRVECWGPLREAVGPLSTTTSSSSSSSLSESTIAEAGEGSNNWEIPKPCLEACACCFPPMSSIPWSHDLVKNQDDDDGATHQGLQQKA

SEQ ID NO: 41 - protein, 290 aa, Pinus taeda
MAVARISAGKTQHCYSFSPSDVRISSAPQNSQSQFKRKSKIKLSSRFLASESSWNGLVAHQLQCNNRHRTNSSFPRSTSRVVARLFGPAIFQASKLKVLFLGTHEEKHPAHLPRTYTLTHSDITAKLTLAFSQTINKDQGWYNRLQRDEVLAQWKKSQGKMSLHVHCHISGGHWLLDAIARLRFYIFRKELPVVLEAFRHGDRALLEKHPELETALVWVYFHSNVKEFKRVECWGSLAEACKGAPSNLNKELDELDGGKLEMPSHCAEPCSCCFPPFSVLLRPEDVEQFS

SEQ ID NO: 42 - protein, 271 aa, Citrus sinensis
MASLVAALGLPSKLKASPYEQQNALFVSRRRSKKKNQSFAPVARLFGPAIFEASKLKVLFLGVDEEKHPGKLPRTYTLTHSDITSKLTLAISQTINNSQLQGWYNRLQRDEVVAEWKKVKGKMSLHVHCHISGGHFLLDICARLRFFIFSKELPVVLKAFVHGDGNLLNNHPELQEALVWVYFHSNIPEFNKVECWGPLKEAVAGSSEAGGTRHEIRQETSISNWELPEPCQETCNCCFPPMSLIPWSEKLPLQTENRGTQGQESLQQQTR

SEQ ID NO: 43 - protein, 263 aa, Medicago truncatula
MGTLTTAPPPMLTSKFKPSFSPQHKPLFPNRRRLWKKNQSIVPVARLFGPAIFEASKLKVLFLGIDEDKHPGNLPRTYTLTHSDVTSKLTLAISQTINNSQLQGWYNRLQRDEVVAQWKKVKGKMSLHVHCHISGGHFLLDIFARLRYFIFCKELPVVLKAFVHGDGNLFNNYPELQEALVWVYFHSKIPEFNKVECWGPLKEASQPTSGTQRDHQNLTLPEPCQETCECCFPPLKLSPMPCSNEVHNDTYEPIDGIETQQSL

SEQ ID NO: 44 - protein, 272 aa, Solanum tuberosum
MGTLTASLVVPSKLNNEKQSSIFVHKTRRKSKKNQSIVPVARLFGPAIFEASKLKVLFLGVDEEKHPGKLPRTYTLTHSDITSKLTLAISQTINNSQLQGWYNRLQRDEVVAEWKKVKGKMSLHVHCHISGGHFMLDLFARLRNYIFCKELPVVLKAFVHGDENLLKNNPELQEALVWVYFHSNIQEFNKVECWGPLKDATSPSSSSSGVGGVKSTSFTSNSNNKWELPKPCEEACACCFPPMSVMPWPSSNLDGIGEENGTIQQGLQEQQS

SEQ ID NO: 45 - protein, 269 aa, Populus tremula x Populus tremuloides
MGSLAIAPFLPSKLRPSILDQNSSLFPSKKKLKRKNQSISPVARLFGPSIFEASKLKVLFLGVDEKKHPGNLPRTYTLTHSDITAKLTLAISQTINNSQLQGWSNKLYRDEVVAEWKKVKGKMSLHVHCHISGGHFLLDLCCRLRYFIFRKELPVVLKAFFHGDGNLFSSYPELQEALVWVYFHSNIPEFNKVECWGPLKHAAAPYTAASGGAPENKEQATDWNLPEPCQENCQCCFPPMSLIPWSEMVPQENKNNPSTQQTFQQAQQP

SEQ ID NO: 46 - protein, 270 aa, Populus tremula x Populus tremuloides
MGSLAVAPFLPSKPRPSLFDQHSSLFSPSTKLKRKNQSISPVARLFGPSIFEASKLKVLFLGVDEKEHPGNLPRTYTLTHSDMTAKLTLAISQTINNSQLQGWSNKLYRDEVVAEWKKVKGKMSLHVHCHISGGHFLLDWCCRLRYFIFRRELPVVLKAFFHGDGSLLSNYPELQEGLVWVYFHSNIPEFSKVECWGPLKDAAAPSTSETGGSNETEELANQSSNWDLPEPCQEENCSCCFPPMSLIPWSKMVPLEDKNNPSTPQNLQQP

SEQ ID NO: 47 - protein, 286 aa, Mesembryanthemum crystallinum
MGTLTASMLLPSKLKPSVFEDQSSVYFKRSCRGLPKLNKAKSFSPVMRLFGPAIFEASKLKVLFLGVDKEKHPGKLPRTYTLTHSDITSKLTLAISQTINNSQLQGWYNQLQRDEVVAEWKKVKGKMSLHVHCHISGGHILLDLFAKLRFYIFCKELPVVLKAFVHGDENLFNNYPELQEAMVWVYFHSNLEEFNKIECWGPLKDAVARNSKKNKNKNKIDFKLSFKEEDDSPDNELEIPETCKEPCTCCFPPTSVIPWSHSALSQGDDLHLSGGTHQGLEQQQQT

SEQ ID NO: 48 - protein, 268 aa, Arabidopsis thaliana
MCSLSAIMLLPTKLKPAYSDKRSNSSSSSSLFFNNRRSKKKNQSIVPVARLFGPAIFESSKLKVLFLGVDEKKHPSTLPRTYTLTHSDITAKLTLAISQSINNSQLQGWANRLYRDEVVAEWKKVKGKMSLHVHCHISGGHFLLDLFAKFRYFIFCKELPVVLKAFVHGDGNLLNNYPELQEALVWVYFHSNVNEFNKVECWGPLWEAVSPDGHKTETLPEARCADECSCCFPTVSSIPWSHSLSNEGVNGYSGTQTEGIATPNPEKL

SEQ ID NO: 49 - protein, 271 aa, Arabidopsis thaliana
MCSLATNLLLPSKMKPVFPEKLSTSSLCVTTRRSKMKNRSIVPVARLFGPAIFEASKLKVLFLGVDEKKHPAKLPRTYTLTHSDITAKLTLAISQSINNSQLQGWANKLFRDEVVGEWKKVKGKMSLHVHCHISGGHFFLNLIAKLRYYIFCKELPVVLEAFAHGDEYLLNNHPELQESPVWVYFHSNIPEYNKVECWGPLWEAMSQHQHDGRTHKKSETLPELPCPDECKCCFPTVSTIPWSHRHYQHTAADENVADGLLEIPNPGKSKG

SEQ ID NO: 50 - protein, 221 aa, Lycopersicon esculentum
MGTLTTSLVVPSKLNNEQQSSIFIHKTRRKCKKNQSIVPVARLFGPAIFEASKLKVLFLGVDEEKHPGKLPRTYTLTHSDITSKLTLAISQTINNSQLQGWYNRLQRDEVVAEWKKVKGKMSLHVHCHISGGHFMLDLFARLRNYIFCKELPVVLKAFVHGDENLLRNYPELQEALVWVYFHSNIQEFNKVECWGPLRDATSPSSSSGGVGGVKSTSFTSH

SEQ ID NO: 51 - protein, 110 aa, Beta vulgaris
PELQEASVWVYFHSSIPEFNKVECWGPLTDAVDPPSKNKKRMMMINDEQDKEEEEEASSSKWEMLVPCTKPCRCCFPPTSLIPWTPSLSQEQQQEQQLPGDVSIPPPGTR

SEQ ID NO: 52 - protein, 187 aa, Zosterops japonica
TYTLTHSDVTAKLTLAVSHTIHAAQLQGWYNRLQRDEVVAEWRKVRGNMSLHVHCHISGGHFLRDLIAPLRYYIFRKELPVVLKAFVHGDGSLFSSHPELEEATVWVYFHSNLPRFNRVECWGPLCDAAAPVEEEGQQNDDRLPAGEWPRRCPQQCECCFPPHSLIPWPNEHDMAPTDAPAAGQTQQ

SEQ ID NO: 53 - protein, 93 aa, Lotus corniculatus
YPELQDALVWVYFHSKIPEFNKVQCWGPLKEAAAPSGGSPEKEGEGVKMPDPCPEECECCFPPPPALDPIPWSEEVPSPHYEAFDGVGTRPNL

SEQ ID NO: 54 - protein, 107 aa, Lotus corniculatus
DLCAKLRYFIFCKELPVVLKAFIHGDENLFNNYPELEESLVWVYFHSNISEFNKVECWGPLKDACATSIGSYSYDKGMPQTQPCQQNCECCFTPMSSSDWIGTQQKL

SEQ ID NO: 55 - protein, 137 aa, Saccharum officinarum
TRLDLIAGLRYYIFRKELPVVLKAFVHGDGDLFSRHPELEDATVWVYFHSNLTRFNRVECWGPLRDAAAPPAEEDSTAPAASNSKEGQMPPVGEWPYRCPQQCDCCFPPHSLIPWPNEHDMAAAAADATAAGQAQQQ

SEQ ID NO: 56 - protein, 159 aa, Picea abies
INKDQLQGWYNRLQRDEVIAQWKKSQGKMSLHVHCHISGGHWLLDAIARLRFYIFRKELPVVLEAFRHGDRALLDKHPELETALVWVYFHSNVREFKRVECWGSLAEACKGAPSNLEKELDEEFNGEKLEMPSHCSEPCNCCFPPFSVLLRPEDAEQFI

SEQ ID NO: 57 - protein, 210 aa, Brassica napus
MCSLATNLLLPSTMKPAFTEKQNTNSLFLTNKRSLMQNRSTVPVPVARLLEPAIFEASKLKVSFLGVDEKKHPSKLPRTYTLTHSDITAKLTLAISQSINNSQLQGWANRLFRDEVVAEWKKVKGKMSLHVHCHISGGHFLLDLIAKLRYYIFCKELPVVLKAFVHGDGNLLNSYPELQESPVWVYSIQTSPSTIRLNVGGRFGRPRSTN

SEQ ID NO: 58 - protein, 97 aa, Brassica napus
MCSLSANMLLPTKLKPAYSDKRGNSTNSLLVSNTRSKRKNQSVVPMARLFGPAIFESSKLKVLFLGVDDKKHPPTLPRTYTLTHSDITAKLTLAISH

(19) United States
(12) Patent Application Publication
(10) Pub. No.: US 2007/0094744 A1
(43) Pub. Date: Apr. 26, 2007

(54) A NOVEL STAY-GREEN GENE AND METHOD FOR PREPARING STAY-GREEN TRANSGENIC PLANTS

(76) Inventor: Nam-Chon Paek, GYEONGGI-DO (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/587,312

(22) PCT Filed: Jan. 12, 2005
(86) PCT No.: PCT/KR05/00104

§ 371 Date: Jul. 26, 2006

(30) Foreign Application Priority Data
Feb. 23, 2004 (KR) 10-2004-0012026

(51) Int. Cl. C12N 15/82; A01H 1/06; A01H 5/00; A01H 1/00; C12N 15/29

(52) U.S. Cl. 800/278; 536/23.6; 530/370; 435/320.1; 435/252.3; 800/298; 800/276; 800/285

(57) ABSTRACT

The present invention relates to a novel SGR (STAY-GREEN) gene participating in chlorophyll catabolism during plant senescence, thereby causing leaf yellowing, a method for preparing stay-green transgenic plants maintaining greenness during leaf senescence, which being characterized by mutating the SGR gene, suppressing the expression of the SGR gene, or deactivating the SGR protein which is encoded by the SGR gene, and stay-green mutant plants produced by said method. According to the present invention, leaf greenness can be maintained for a long time by mutating the SGR gene, suppressing the expression of the SGR gene, or inactivating the protein encoded by the SGR gene, thereby preventing leaf yellowing of plants in yellowing plant caused by chlorophyll catabolism during leaf senescence.

### TECHNICAL FIELD

The present invention relates to a novel SGR (STAY-GREEN) gene participating in chlorophyll catabolism during plant senescence, thereby causing leaf yellowing, a method for producing stay-green transgenic plants, which being characterized by mutating the SGR gene, suppressing the expression of the SGR gene, or deactivating the SGR protein which is encoded by the SGR gene, and stay-green transgenic plants produced by said method.

### BACKGROUND ART

Senescence is the final stage of growth and development in plants. Leaf yellowing due to chlorophyll degradation is widely used as a phenotypic marker of the initiation of plant senescence. Although leaf senescence is induced by a number of environmental and developmental factors, the timing of leaf senescence is controlled by programmed cell death process according to the genetic background rather than a passive degenerative process.

Many senescence-related mutants have been found in crop plants that maintain leaf greenness after the grain ripening stage, and which are referred to as stay-green mutant or non-yellowing mutant.

The mutants showing stay-green phenotype are classified into five types according to their characteristics as shown FIG. 1 (Thomas, H. & Howarth, C. J., J. Exp. Bot., 51:329, 2000). With type A stay-green, the initiation of senescence is much delayed, but once senescence is started, it proceeds at the same rate as the wild type. Type B stay-green initiates senescence at the same time period as the wild type, but leaf yellowing and the decrease in photosynthetic rate caused by senescence proceed slowly. The above two types are regarded as functional stay-green due to the prolonged photosynthetic activity during seed filling.

On the other hand, type C stay-green retains chlorophyll almost indefinitely during senescence due to genetic malfunction of chlorophyll degradation mechanisms. However, as senescence proceeds normally in plant tissues in terms of the physiological function, it is called ‘nonfunctional stay-green or ’cosmetic stay-green. The leaf death by abrupt freezing or drying results in type D stay-green. Finally, type E stay-green accumulates higher chlorophyll content in leaves, thus maintaining dark green, but without increasing the photosynthesis rate.

Thus, the functional stay-green (types A and B) retains both high chlorophyll content and photosynthetic competence in leaves during seed filling, while leaves of the nonfunctional stay-green (types C, D and E) appear green but the photosynthetic competence is almost the same as the wild type.

Physiological, cytological, biochemical and genetic features of the non-yellowing mutant derived from a forage grass Festuca pratensis have been reported in detail (Thomas, H., Planta, 137:53, 1977; Thomas, H., Planta, 154:212, 1982; Thomas, H., Theor. Appl. Genet., 73:551, 1987; Thomas, H. & Matile, P., Phytochemistry, 27:342, 1988). The stay-green trait of Festuca pratensis was nonfunctional and induced by natural mutation and regulated by a single recessive allele of the nuclear locus, sid.

In soybean, the stay-green mutant was controlled by three nuclear genes G and dld2, and a cytoplasmic gene cytG (Guiamet, J. J. et al., Plant Cell Physiol., 31:1123, 1990). A dominant gene, G keeps the seed coats green. A cytoplasmic gene, cytG and two recessive alleles, dldld2d2 and G_dldld2d2, regulate the greenness in leaves, pod walls, seed coats, and embryos. It is reported that the cytG mutation renders the chlorophyll b more stable than chlorophyll a in senescing soybean leaves, thus suppressing chlorophyll degradation, and the dldld2d2 homozygous mutation shows a significant delay of degradations of chlorophyll and soluble rubisco protein during senescence in soybean leaves (Guiamet, J. J. et al., Plant Physiol., 96:227, 1991; Guiamet, J. J. et al., Physiol. Plant., 96:655, 1996).

There has been a report that the amount of leaf chlorophylls decreased in the yellowing kidney bean (Phaseolus vulgaris) cultivar, Red Mexican during leaf senescence, while the stay-green cultivar, Alamo, retained the chlorophylls in the senescent leaves (Bachmann, A. et al., New Phytol., 126:593, 1994). In Phaseolus vulgaris, chlorophyllase activity of the non-yellowing mutant leaves was not altered. Among chlrorophyll degradation products, pheophorbide a representing green was detected neither in the senescent leaves of the wild type nor those of the mutant.

However, accumulation of chlorophyllide a and b was detected in the non-yellowing mutant, while no detectable amount was present in the wild type. It indicates that chlorophyllides are not quickly catabolized during senescence of the mutant leaves, thus reporting that the mutant may be deficient in Mg-dechelatase activity (Fang, Z. et al., J. Exp. Bot., 49:503, 1998).

It was reported that the stay-green phenotypes of soybean and the Festuca pratensis stay-green mutants were due to the reduced activity of phaeophobide a oxygenase (PaO) during chlorophyll catabolism. However, a recent report demonstrates that the mutant (acd1:accelerated cell death 1) without PaO function in Arabidopsis does not show stay-green phenotype (Tanaka, R. et al., Plant Cell Physiol., 44:1266, 2003).

Until now, several reports have described the physiological and phenotypic characteristics with respect to different types of stay-green plants induced spontaneously or artificially. In previous research, the present inventors disclosed that the stay-green mutant induced by the treatment with a chemical mutagen MNU in glutinous rice Hwacheong-wx is similar to the type C mutant (FIG. 1), transmitted by a single recessive nuclear gene and the mutant gene is named sgr (stay-green) and found that the sgr locus is located on the long arm of chromosome 9 (Cha, K. W. et al., Theor. Appl. Genet., 104:526, 2002).

However, the sequence of the wild type SGR gene and the sgr mutant gene, and the amino acid sequence and the function of the SGR protein encoded by the SGR gene have not yet been revealed.

Accordingly, the present inventors have made an effort to find which gene mutation on chromosome 9 caused the stay-green phenotype, and finally succeeded in discovering the new SGR gene involved in chlorophyll degradation on chromosome 9 of rice during leaf senescence. Mutation site of the sgr mutant gene, at which the gene recessive mutation occurred, by map-based gene cloning, confirmed that stay-green property of the stay-green mutant is due to SGR gene mutation. And also, the present inventors confirmed that stay-green mutant plant can be produced by suppressing the expressions of 2 SGR genes (AtSGR1 and AtSGR2) which are strongly expressed during leaf senescence in Arabidopsis through RNAi (RNA interference) gene silencing transgenic method, thereby completing the present invention.

### DISCLOSURE OF THE INVENTION

An object of the present invention is to provide a novel SGR gene participating in chlorophyll catabolism during leaf senescence, thereby causing leaf yellowing and a polypeptide encoded by the gene.

Another object of the present invention is to provide a method for producing a stay-green mutant plant, which comprises mutating the SGR gene and a stay-green mutant plant produced by the method.

Still another object of the present invention is to provide a method for producing a stay-green mutant plant, the method being characterized by suppressing the expression of the SGR gene, or deactivating the protein which is encoded by the SGR gene, and stay-green transgenic plant produced by the method.

To achieve the above objects, in one aspect, the present invention provides a SGR gene encoding a polypeptide comprising amino acid sequence region having at least 60% homology with SGR domain I which is conserved amino acid sequence region of 49˜207 among 274 amino acid sequences of SEQ ID NO: 30, and triggering leaf yellowing by participating in chlorophyll catabolism during plant senescence.

In the present invention, the polypeptide comprises a chloroplast-targeting signal peptide sequence, SGR domain II in N-terminus, and/or SGR domain III in which 2˜6 glutamines (Qs) are conserved in C-terminal region, more preferably, the polypeptide comprises the amino acid sequence selected from the group consisting of SEQ ID NOs: 30 to 50 and 57. The SGR gene comprises the DNA sequence selected from the group consisting of SEQ ID NOs: 1 to 21 and 28.

In another aspect, the present invention provides polypeptide encoded by the SGR gene.

In still another aspect, the present invention provides recombinant vector comprising the SGR gene, microorganism transformed with the recombinant vector and plant transformed with the SGR gene.

In yet another aspect, the present invention provides a method for producing a stay-green mutant plant, the method comprises mutating the SGR gene or fragments thereof and a stay-green mutant plant produced by the method. The mutation of SGR gene in the present invention is comprised of deleting a part of base of said gene, substituting other singular or plural bases for a part of base of said gene, or adding other singular or plural bases to said gene, more specifically, a substitution of A for the 295^{th} base G in SEQ ID NO: 1 SGR gene.

In yet another aspect, the present invention provides a method for producing a stay-green mutant plant, the method comprises suppressing the expression of the SGR gene in a yellowing plant and a stay-green mutant plant produced by the method. The suppression of the expression of the SGR gene in the present invention is preferably performed by gene silencing technique.

In yet another aspect, the present invention provides a method for producing a stay-green mutant plant, the method comprising the steps of: (a) obtaining a gene silencing recombinant vector by introducing a SGR gene or a fragment thereof originated from target plant to be mutated, to T-DNA vector; and (b) transforming a wild type plant with the recombinant vector and a stay-green mutant plant produced by the method. The presentive T-DNA vector is preferably a vector for RNAi which induces gene silencing by making a double-stranded RNA (dsRNA) in transgenic plants, the recombinant vector preferably comprises CaMV35S promoter or senescence-enhanced promoter.

In yet another aspect, the present invention provides a method for producing a stay-green mutant plant by means of deactivating the protein encoded by the SGR gene in yellowing plants during leaf senescence, and a stay-green mutant plant produced by the method.

In the method for producing a stay-green mutant plant according to the present invention, the SGR gene encodes the polypeptide comprising amino acid sequence having at least 60% homology with SGR domain I which is conserved amino acid sequence region of 49˜207 among amino acid sequence of SEQ ID NO: 30, more specifically, the SGR gene comprises the DNA sequence selected from the group consisting of SEQ ID NOs: 1 to 21 and 28. And also, the SGR gene fragment comprises the DNA sequence selected from the group consisting of SEQ ID NOs: 21 to 29.

In the present invention, the stay-green sgr mutant gene is the general term of having the function of keeping greenness without leaf yellowing even during senescence by losing the function of the wild type SGR gene. The sgr mutant gene is obtained by deleting a part of base of said gene, substituting other singular or plural bases for a part of base of said gene, or adding other singular or plural bases to said gene.

### BRIEF DESCRIPTION OF DRAWINGS

FIG. 1 is a graphic diagram showing photosynthesis efficiency and the change of chlorophyll concentration of leaves with the passage of time, according to the type of stay-green mutant expressing stay-green relatively when compared with the wild type during senescence.

FIG. 2 is a photograph showing the phenotypic difference (the change of leaf color and chlorophyll concentration caused by senescence) between the leaves of wild type (left) and sgr mutant (right) in rice. A represents the onset of heading, B represents after natural senescence proceed during grain filling, C represents the color difference in the detached leaves of one month old rice, appeared after inducing senescence artificially by dark treatment for 9 days at room temperature. D represents the result of measurement performed every 2 days for 10 days on the changes in chlorophyll concentrations of the detached leaves of one-month-old sgr mutant and the wild type after dark treatment.

FIG. 3 is a graphic diagram showing the result of the measurement of the nitrogen content of the plant and the seed respectively by separating the seed and the plant after the rice was harvested at 40 days after heading, which the seed maturity is complete.

FIG. 4 is a graphic diagram showing the comparison of changes in the chlorophyll concentration between the stay-green mutant and the wild type of rice according to leaf senescence after heading.

FIG. 5 is a graphic diagram showing the comparison of changes in the photosynthetic rate between the stay-green mutant and the wild type of rice according to leaf senescence after heading.

FIG. 6 is a molecular genetic map showing that the sgr gene of the stay-green mutant is located on the long arm of chromosome 9 in rice.

FIG. 7 represents the result of comparison and analysis by HPLC using the standard material of the chlorophyll of the leaves and the catabolic intermediates after dark-induced senescence for 6 day in the leaves of the stay-green mutant and the wild type (Hwacheongchalbyeo:Hwacheong-wx).

FIG. 8 represents the result of western blotting analysis showing the change of the concentration of protein LHCP I (light-harvesting chlorophyll a/b-binding protein I) and subunit proteins of LHCP II which are attached to thylakoid membrane of chloroplast and contain chlorophylls, D1, psaA/B, and cyrochrome protein Cytb₆f without chlorophylls and Rubisco large subunit (rbcL) protein located in stroma of chloroplast with the passage of time during dark-induced senescence at 1 (control), 3, 5, 7 day dark treatment on the leaves of the stay-green mutant and the wild type of rice.

FIG. 9 is a photograph of ultrastructural TEM analysis of chloroplast degradation process after inducing senescence by dark treatment of the leaves of the wild type (a, c, e) and the stay-green mutant (b, d, f, g, h) of rice. The a and b represent normal chlorophyll of the wild type (left) and the stay-green mutant (right) respectively, right before dark treatment, c and d represent chlorophyll after 6 day dark treatment, e and f represent cells of wild type (left) and stay-green mutant (right) cells after 9 day dark treatment. The g and h is a photograph of conserved thylakoid membrane of chloroplast conserved without degradation in the sgr mutant cell, which is an enlargement of g and h region in photograph f.

FIG. 10 is the fine mapping of the molecular genetic map of the stay-green sgr mutant, indicating that the normal SGR gene DNA mutation in stay-green mutant genomic DNA is located within 33.9 kb and 38.2 kb (total 4.3 kb) in AP005314 DNA sequence (about 140 kb) which comes from a PAC clone of the wild type japonica cultivar (Nipponbare).

FIG. 11 shows the result of the comparison using AP005314 DNA sequence as a template after sequencing 4.3 kb DNA sequence region represented in FIG. 10 among AP005314 DNA sequences which is a PAC clone in DNA library of japonica type (Nipponbare) by PCR-amplifying the genome DNA between the wild type (Hwacheong glutinous japonica rice and indica-type Milyang23 rice) and the stay-green mutant derived from Hwacheong glutinous rice. Boxes indicate the locus that a single base substitution occurs from guanine (G) to adenine (A), so that a wild type SGR gene is transformed into a stay-green sgr mutant gene.

FIG. 12 shows a substitution of methionine (M:atg) for the 99^{th} valine (V:gtg) of the wild type SGR protein in the sequence of the wild type SGR gene (SEQ ID NO: 1; GenBank Accession NO:AY850134), the 274 amino acid sequence deduced from the SGR gene, and the protein encoded with the sgr mutant gene inducing stay-green phenotype.

FIG. 13 shows the result of a northern blotting indicating that the expression level of the wild type SGR gene remains high from 2^{nd} day after dark treatment to 8^{th} day when leaf-yellowing is complete in the case of dark-induced senescence for 0 (control), 2, 4, 6 and 8 days in the wild type rice, Hwacheong-wx.

FIG. 14 is a photograph of a northern blotting showing the suppression of the wild type SGR gene expression, in the case of suppressing senescence by treating with 100 μM 6-benzylaminopurine (6-BA) which is a precursor of cytokinin, a plant senescence-suppressing hormone, during 2-day dark treatment of the wild type rice.

FIG. 15 shows the result of a southen blotting using the wild type SGR gene as a probe after treating genome DNA of japonica type stay-green mutant (1 lane), indica-type Milyang23 (2 lane) and F₁ hybrid plant (3 lane) obtained by crossing the two of them, with a restriction enzyme DraI.

FIG. 16 is a boxshade form which represents the comparison of polypeptides of ZmSGR1 (SEQ ID NO: 34) and ZmSGR2 (SEQ ID NO: 35) of Maize, TaSGR1 (SEQ ID NO: 32) and TaSGR2 (SEQ ID NO: 33) of Wheat, HvSGR (SEQ ID NO: 31) of Barley, SbSGR (SEQ ID NO: 36) of Sorghum, which are a monocotyledon, among angiosperm having homology with SGR amino acid sequence (OsSGR) in rice (SEQ ID NO: 30).

FIG. 17 is a boxshade form which represents the comparison of polypeptides of GmSGR1 (SEQ ID NO: 37) and GmSGR2 (SEQ ID NO: 38) of Soybean, VvSGR (SEQ ID NO: 39) of Grape, LsSGR (SEQ ID NO: 40) of Lettuce, CsSGR (SEQ ID NO: 42) of Orange, MtSGR (SEQ ID NO: 43) of Medicago truticatula, StSGR (SEQ ID NO: 44) of Potato, PoSGR1 (SEQ ID NO: 45) and PoSGR2 (SEQ ID NO: 46) of Poplar, McSGR (SEQ ID NO: 47) of Iceplant which is a composite, AtSGR1 (SEQ ID NO: 48; At4g22920) and AtSGR2 (SEQ ID NO: 49; At4g11910) of Arabidopsis thaliana, which are a dicotyledon among angiosperm having homology with SGR amino acid sequence (OsSGR) in rice (SEQ ID NO: 30).

FIG. 18 is a boxshade form which represents the comparison between PtSGR (SEQ ID NO: 41) amino acid sequence of Pine which is a gymnosperm having homology with SGR polypeptide in rice (SEQ ID NO: 30), and SGR polypeptides of Rice and Orange.

FIG. 19 represents each domain in a solid line, box, dotted line, double solid line forms by comparing SGR polypeptides of other species plants which have homology with SGR polypeptide of Rice (SEQ ID NO: 30).

FIG. 20 is a boxshade form which represents the comparison between the partial sequence of protein of LsSGR (SEQ ID NO: 50) of Tomato, BvSGR (SEQ ID NO: 51) of Sugarbeet, LjSGR1 (SEQ ID NO: 53) and LjSGR2 (SEQ ID NO: 54) of Lotus japonicus, BnSGR1 (SEQ ID NO: 57) and BnSGR2 (SEQ ID NO: 58) of Rape which have high homology with SGR protein among genes of dicotyledon wherein only partial DNA sequence is decoded, and SGR polypeptide of Potato (SEQ ID NO: 44).

FIG. 21 is a boxshade form representing the comparison between the partial sequence of protein of ZjSGR (SEQ ID NO: 52) of Zoysia japonica and SoSGR (SEQ ID NO: 55) of Sugarcane which are high homologous with SGR protein among genes of monocotyledon wherein only partial DNA sequence is decoded, and SGR polypeptide of Rice (SEQ ID NO: 30).

FIG. 22 is a boxshade form representing the comparison between the partial sequence of protein of PiSGR (SEQ ID NO: 56) of Spruce which has high homology with SGR protein among genes of gymnosperm wherein partial DNA sequence is decoded, and SGR polypeptide of Pine (SEQ ID NO: 41).

FIG. 23 shows the result of RT-PCR performed on RNA obtained from 0 (control), 1, 3, 6 and 9-day dark-treated mature leaves of Arabidopsis using primer prepared from At4g22920 and At4g11910 gene sequence of Arabidopsis.

FIG. 24 is a photograph of SGR protein (OsSGR) of Rice and one of 2 SGR proteins of Arabidopsis, AtSGR1 (At4g22920) protein, accumulated in chloroplast after synthesis in ER in protoplast of the leaf of Arabidopsis by means of protoplast transient assay with CaMV35S::SGR-GFP.

FIG. 25 is a photograph representing the result of inducing senescence for 0, 3 and 7 day dark treatment on detatched mature leaves of control and leaves (T1-1) obtained by the steps wherein PCR cloning is performed on a site with the highest homology obtained by comparing DNA base sequences between At4g22920 and At4g11910 which are two SGR genes of Arabidopsis, using At4g11910 cDNA (AtSGR2) as a template, and then RNAi T-DNA vector producing hairpin dsRNA (double-stranded RNA) is prepared by ligating the 480-bp At4g11910 cDNA fragment into a pB7GWIWG2(II) vector which induces RNA interference gene silencing, followed by transforming it into the wild type (Col-0) of Arabidopsis.

FIG. 26 is a photograph of showing the expression levels of the At4g11910 and the At4g22920 genes by RT-PCR performed on RNA extracted from 3-day dark-treated wild type leaves (control: Col-0) and the RNAi gene silencing transgenic leaves of the T1-1 (3DAD leaves of the FIG. 22). EF1a is a gene of elongation factor 1a of Arabidopsis which is a control, indicating the amount of cDNA used in PCR is the same each other. DAD represents days after dark treatment.

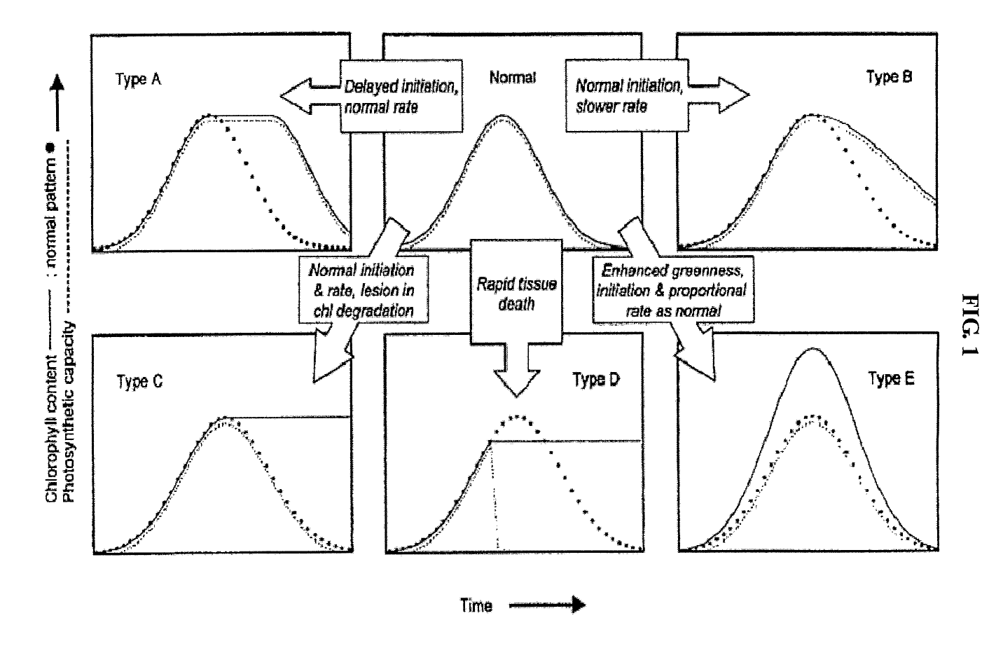

### DETAILED DESCRIPTION OF THE INVENTION

The present invention will hereinafter be described in further detail by examples. It will, however, be obvious to a person skilled in the art that these examples are given for illustrative purpose only, and the scope of the present invention is not limited to or by these examples.

Particularly, although it is described in the following examples only on the essential SGR gene for triggering chlorophyll catabolism during plant senescence, and the stay-green sgr mutant plant and gene whose the function is lost by MNU mutagenesis, and the mutant plant in which SGR gene expression is suppressed, it is also within the scope of the present invention to prepare recombinant vectors comprising the SGR gene, microorganisms transformed with the recombinant vector and transgenic plants by using said gene information.

Furthermore, although a method for producing stay-green mutant plant by using a model plant, Arabidopsis thaliana, was illustrated in the present invention, it is also within the scope of the present invention to produce stay-green mutant plant in any plant containing SGR gene and having yellowing phenotype during senescence. And, it is also obvious to a person skilled in the art to produce stay-green mutant plant by inactivating the protein encoded by SGR gene in yellowing plants during senescence. Although the mutant plant propagates by sexual reproduction, it is obvious to a person skilled in the art that it can be reproduced repeatedly and asexually by tissue culture etc.

### EXAMPLE 1

### Induction of the Stay-Green Mutant Rice by the Treatment of MNU (N-methyl N-nitrosourea)

The stay-green mutant was induced by the treatment of a chemical mutagen N-methyl N-nitrosourea (MNU) in the fertilized egg cells of a glutinous rice Hwacheong-wx which is a japonica rice cultivar.

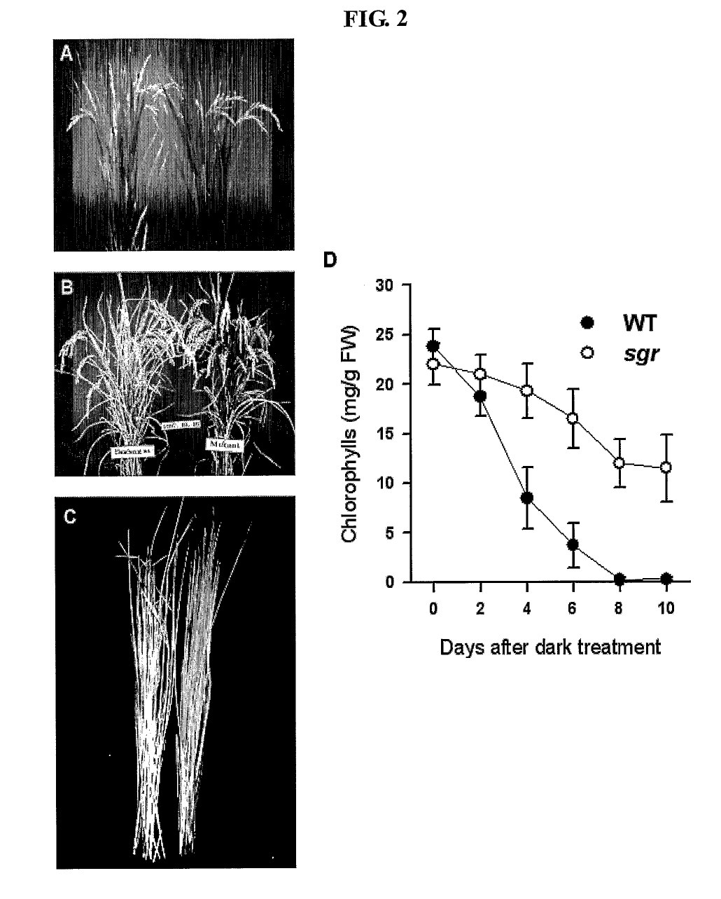

About 30,000 M2 seeds were harvested from M1 plants obtained by growing about 1,500 MNU (N-methyl N-nitrosourea)-treated M1 seeds of the glutinous japonica rice Hwacheong-wx. A stay-green mutant showing a green leaf phenotype even after grain filling was identified and isolated among M2 plants grown from the M2 seeds. During vegetative growth of the rice before heading, no phenotypic difference was observed between the wild type and the stay-green mutant plants (FIG. 2A).

The sgr mutant leaves remained green while the wild type leaves turned yellow gradually due to leaf senescence during grain filling (FIG. 2B). Also, in the case of dark-induced senescence by 2-week dark treatment on detatched leaves, the sgr mutant leaves maintained greenness, while the wild type leaves turned yellow completely. The phenotype of the detached leaves after 9 day dark treatment (FIG. 2C) at any growth stage of rice was consistent with that of naturally senesced leaves (FIG. 2B). The result of analyzing the change of chlorophyll concentration every 2 days during dark treatment shows the tendency of the suppression of chlorophyll degradation in the sgr mutant, thus indicating that the stay-green trait of the sgr mutant is due to much slower rate of chlorophyll degradation (FIG. 2D).

### EXAMPLE 2

### Phenotypic and Physiological Characteristics of the Stay-Green Mutant Rice

The stay-green mutant didn't show any significant difference from the wild type on phenotypic traits such as heading date, culm and panicle lengths, panicle number per hill, spiklet number per panicle, fertility, 1,000 grain weight, grain dimension, and yield per hill (Table 1).

TABLE 1
Comparison of agricultural characteristics between the wild type and the
stay-green mutant

| | | | | | | | 1000 | | | | |
|---|---|---|---|---|---|---|---|---|---|---|---|
| | | Culm | Panicle | Panicles/ | Spikelets/ | | Grain | Grain Size | | | Yield/ |
| | Heading | Length | Length | Hill | Panicle | Fertility | Weight | Length | Width | Thickness | Hill |
| Line | Date | (cm) | (cm) | (No.) | (No.) | (%) | (g) | (mm) | (mm) | (mm) | (g) |
| Hwecheongloyeo | 8/17 | 89.0 | 18.7 | 17.3 | 129.0 | 92.9 | 21.7 | 4.7 | 2.7 | 1.9 | 45.0 |
| Hwecheong-wx | 8/18 | 93.7 | 21.4 | 17.0 | 126.7 | 91.2 | 20.3 | 4.7 | 2.7 | 1.9 | 42.6 |
| Stay-green | 8/18 | 93.3 | 20.8 | 17.5 | 129.5 | 92.7 | 20.5 | 4.7 | 2.7 | 1.9 | 43.0 |
| Difference | NS | NS | NS | NS | NS | NS | NS | NS | NS | NS | NS |

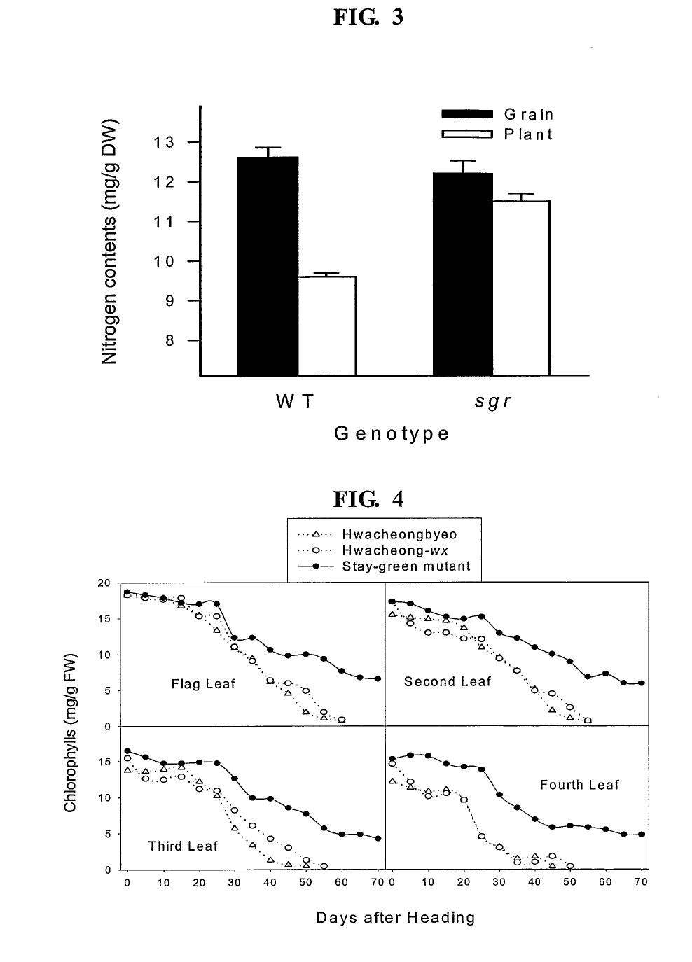

Namely, as described in the Table 1, except for the stay-green trait, various agronomic characteristics of the mutant were similar to the wild type. The only significant difference is between nitrogen content of seed and that of plant (leaf and stem) after harvest. It is considered due to the fact that protein degradation proceeds slowly in the sgr mutant leaves, so that the nitrogen recycling of the mutant from leaves to seeds becomes less efficient than that of the wild type during about 40-day grain filling period after heading (FIG. 3).

The changes in chlorophyll concentration caused by leaf senescence in the stay-green mutant and the wild type during panicle ripening after heading (FIG. 4) was investigated. Leaf chlorophyll was extracted with 80% acetone and the concentration was measured using a spectrophotometer. As a result, as described in FIG. 4, chlorophyll concentration of the mutant and the wild type didn't show any significant difference until heading, but while the panicle ripened, the difference between chlorophyll contents of the wild type and the mutant became apparent so that the wild type turned yellow, and, the mutant remained green even at an early winter temperature with frost, past 50 days after heading.

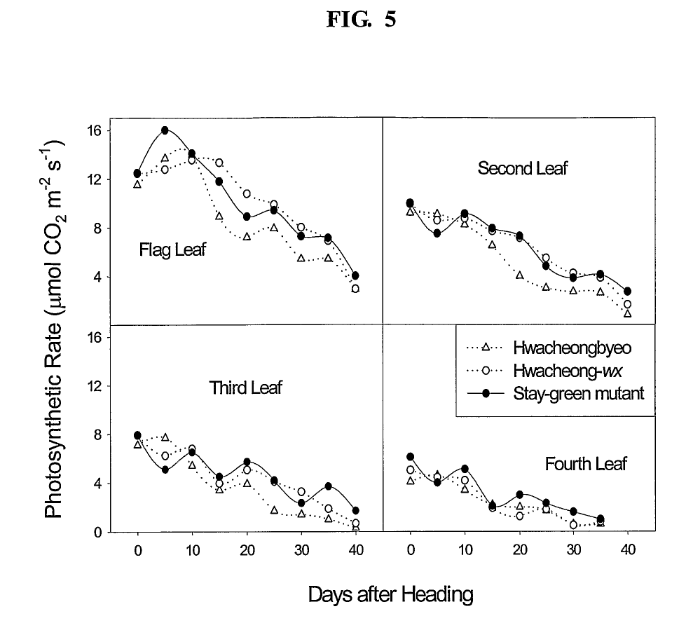

The photosynthetic rate was measured using LI-6400 (Li-Cor, USA) under a fixed LED light source (1,000 μmole·m⁻²·s−1) at 25° C. Means and standard deviations were obtained from at least three replicates and compared using Fisher's LSD (FIG. 5). As described in FIG. 5 the reduction rate of chlorophyll concentration was much slower in the mutant leaves than that in the wild type. However, the photosynthetic rate of green leaves in the mutant was not significantly different from that of yellowing leaves in the wild type during panicle ripening.

### EXAMPLE 3

### Molecular Genetic Mapping by Using Phenotypic and Molecular Markers

The present inventors disclosed that the sgr mutant gene was inherited by a single recessive gene and the mutation locus is located on the long arm of chromosome 9 in rice by using phenotypic and molecular markers (Cha, K. W. et al., Theor. Appl. Genet., 104:526, 2002).

To create the F₂ mapping population to find out the locus on chromosome of the stay-green sgr mutant gene which chlorophyll degradation is suppressed during senescence, the stay-green mutant was crossed with a tongil rice cultivar, wild type Milyang23 which was bred from an indica X japonica hybridization and had a genetic makeup close to indica, thus extracting genomic DNA from the mature leaves of 305 F₂ plants and the parents.

Linkage analysis using phenotypic markers showed that the sgr mutant gene was linked to Dn-1 (Dense panicle-1) gene on chromosome 9 with approximately 25% recombination value. The candidate molecular markers which are presumably around the sgr mutant gene locus were chosen and mapped by surveying the web database (RiceGenes, USA: http://genome.cornell.edu/rice and Rice Genome Research Program, Japan: http://rgp.dna.affrc.gojp) on chromosome 9.

Between the stay-green mutant and Milyang23 as the wild type, two SSR (simple sequence repeat) markers, RM160 and RM189, showed polymorphic bands in approximately 0.1 kb and 0.13 kb regions, respectively. One STS (sequence tagged site) marker, T4, was useful for detecting polymorphism through the presence of a single 0.6 kb band in the stay-green mutant and the absence of it in Milyang23 by PCR.

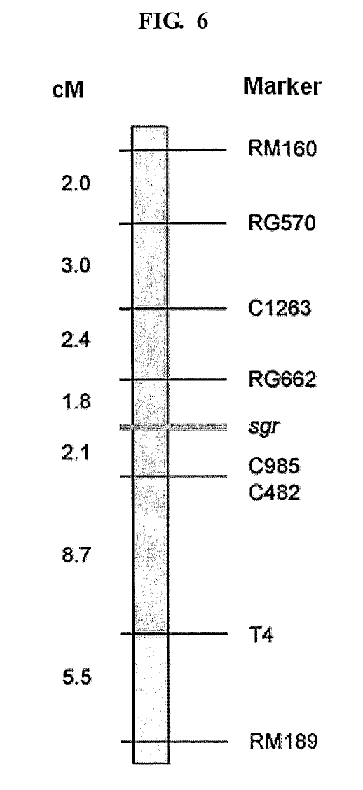

305 F₂ genomic DNA was analyzed using RFLP markers, RG662 (DraI), RG570 (EcoRV), C1263 (DraI) and C482 (EcoRI), which shows polymorphism between the sgr mutant and Milyang23 genomic DNA by restriction enzyme among the eight RFLP markers on chromosome 9, as probes to complete genetic map showing sgr mutant gene locus (FIG. 6).

Using MAPMAKER/EXP 3.0 (Lander, E. S. et al., Genomics, 1:174-81, 1987), the sgr mutant gene was mapped between RG662 and C482 markers, with distances of 1.8 cM and 2.1 cM, respectively, on the long arm of chromosome 9 (Cha, K. W. et al., Theor. Appl. Genet., 104:526, 2002).

### EXAMPLE 4

### HPLC Analysis of the Senescing Leaves of the sgr Mutant

The key chlorophyll-catabolizing enzymes, chlorophyllase, Mg-dechelatase, and Pheophorbide a oxygenase (PaO), are highly activated in the senescing leaves. In the non-yellowing mutant of Phaseolus vulgaris, chlorophyllide accumulates in senescing leaves while undetectable in those of the wild type, thus suggesting that the function of Mg-dechelatase is lost or weakened in the mutant. In the non-yellowing mutant of forage grass Festuca pratensis, PaO activity is lower than that in the wild type, thus suggesting that the activity of PaO is lost or weakened in the mutant.

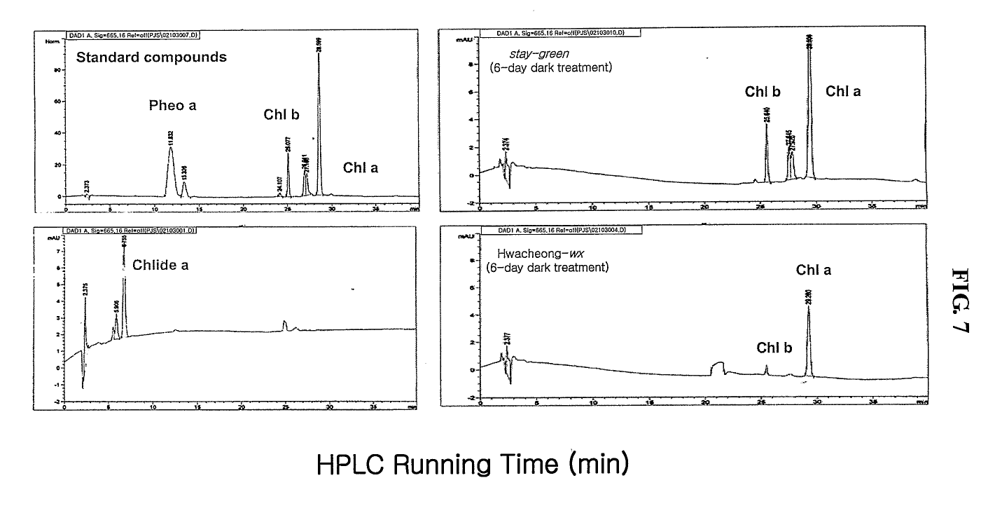

Therefore, in this Example, the chlorophyll content of the senescing leaves in the stay-green mutant rice was investigated. FIG. 7 shows the results of HPLC analysis of chlorophyll contents of the leaves and the intermediate catabolic compounds at 6 days after dark treatment to induce senescence of the presentive stay-green mutant rice and the wild type rice. In FIG. 7, Chl a and Chl b represent chlorophyll a and chlorophyll b, respectively, Chlide a and Pheo a represent chlorophyllide a and pheophorbide a, respectively.

As described in FIG. 7 and FIG. 2D, HPLC analysis of the stay-green mutant of rice shows that the content of chlorophyll in the sgr mutant decreases much more slowly than that of the wild type (Hwacheong glutinous rice; Hwacheong-wx) in the senescing leaves and no intermediate compound, caused by chlorophyll degradation, accumulates. Therefore, the stay-green sgr mutant is not the mutation of the Mg-dechelatase nor PaO gene, moreover, since chlorophyllase of the rice exists on chromosome 10 as one copy it is revealed that the wild type SGR gene is not an enzyme participating directly in chlorophyll catabolism.

### EXAMPLE 5

### Western Blotting Analysis on the Senescing Leaves of the sgr Mutant by Dark Treatment

It has already reported that LHCP II (light-harvesting chlorophyll a/b-binding protein II) which chlorophyll concentration is reduced rapidly in the wild type senescing leaves exists stably in the non-yellowing mutants.

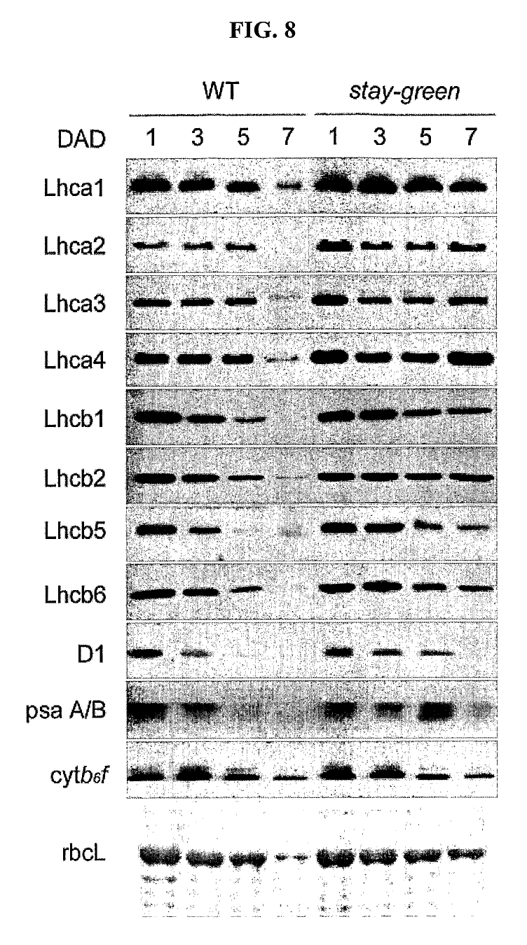

Therefore, in this Example, the change of the subunit proteins of LHCP I (light-harvesting chlorophyll a/b-binding protein I) and LHCP II which are attached on thylakoid membrane of chloroplast and contain the chlorophylls, D1, psaA/B and cytochrome protein Cytb₆f which don't contain chlorophyll, with the passage of time for dark treatment was investigated by western-blotting analysis after inducing senescence by dark treatment on the leaves of the stay-green mutant and the wild type of rice (FIG. 8).

As described in FIG. 8, among thylakoid membrane proteins, the subunits (Lhca1, Lhca2, Lhca3 and Lhca4) of LHCP I and subunits (Lhcb1, Lhcb2, Lhcb5 and Lhcb6) of LHCP II were extremely stable in the senescing leaves of the sgr mutant during dark-induced senescence while other proteins such as D1, Cytb₆F and psaA/B which are also thylakoid membrane proteins without chlorophyll were degraded as normally as the wild type. As a result of that it is concluded that the SGR protein is required for the proteolysis of LHCP I and II subunits during leaf senescence. And also, the degradation of Rubisco large subunit which is an important water-soluble protein in stroma of chloroplast in plant is greatly inhibited, so the stay-green mutant contains the soluble Rubisco proteins in the leaves relatively higher, even though senescence proceeds.

From the above results, the reduction of the chlorophyll degradation of the stay-green mutant inhibits the degradation of LHCP I and LHCP II which contain chlorophyll and also rubico proteins, thereby presuming that the nitrogen content of leaves, namely, the protein content in the sgr mutant leaves is maintains relatively higher after seed harvesting as described in FIG. 3.

### EXAMPLE 6

### Ultrastructural TEM Analysis of the Senescing Leaves of the sgr Mutant by Dark Treatment

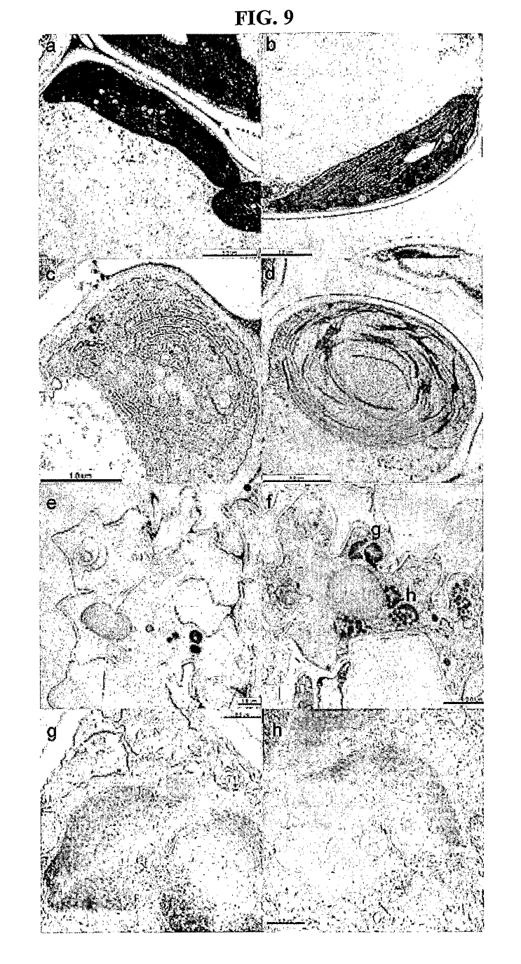

The chloroplast degradation in the senescing leaves of the sgr mutant was analyzed by ultrastructural TEM (FIG. 9). In FIG. 9, a and b represent normal chloroplasts of the wild type (left) and the stay-green mutant (right) right before the dark treatment, respectively, c and d represent the chloroplasts at 6 days after dark treatment (6 DAD), e and f represent the wild type (left) and stay-green mutant (right) cells at 9 DAD, and g and h represent the enlarged TEM photograph of g and h region in photograph f.

As described in FIGS. 9c and 9d, although the chloroplast degradation in the senescing leaves of the sgr mutant occurred at 6 DAD, significant difference from the wild type was observed. As a result of analyzing the leaves of the wild type and the sgr mutant after inducing senescence at 9 DAD, the thylakoid membranes without grana structure (g and h) and chloroplast membranes were observed in the mutant leaf cells, whereas chloroplasts of the wild type disappeared completely. It appears that the persistence of thylakoid membrane structure in the sgr mutant during leaf senescence is related to the stability of LHCP I and II subunits attached to the thylakoid membrane to embrace chlorophylls.

### EXAMPLE 7

### Cloning of SGR Gene and Stay-Green sgr Mutant Gene

The sgr mutant gene was cloned using the genetic map prepared in the Example 3 as a basic material. As described in the genetic map the sgr mutant gene was mapped to 3.9cM interval between RFLP markers, RG662 and C482 on the long arm of chromosome 9.

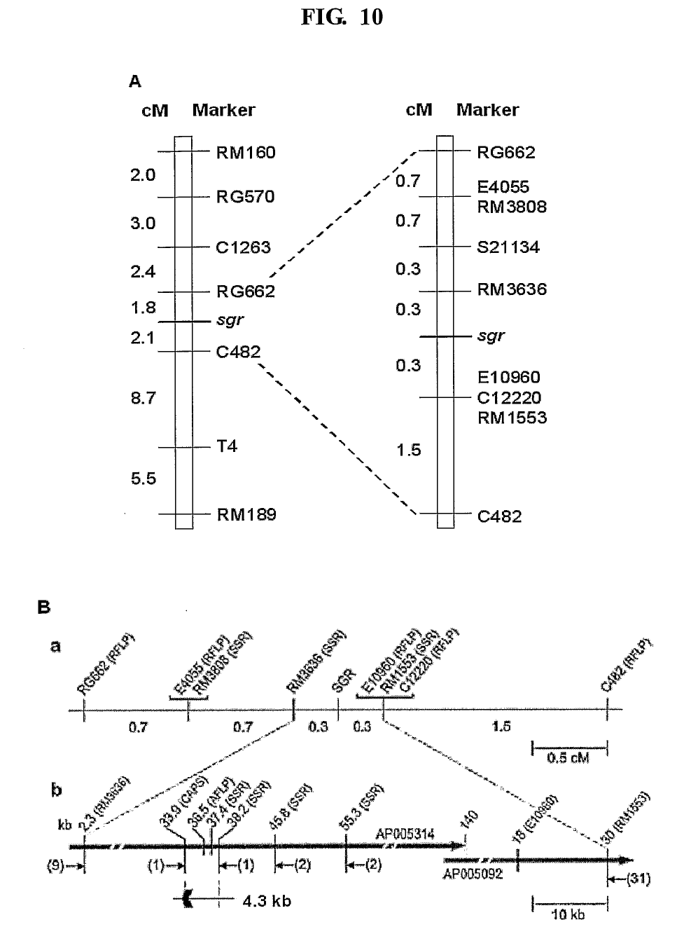

As a result of fine mapping with SSR and RFLP markers, the locus of sgr mutant gene was further mapped to 0.6cM interval between a SSR marker, RM3636 and an RFLP marker, E10960 (EcoRI) (FIG. 10A). The 150 kb genomic region between the RM3636 and E10960 markers have been sequenced from the PAC (AP005314) and BAC (AP005092) clones obtained from the genome of japonica Nipponbare cultivar (Rice Genome Research Program, Japan: http://rgp.dna.affrc.gojp). It is suggested that this genomic region includes 13 genes by gene annotation, one of which is presumed as the sgr mutant gene.

For the fine mapping of sgr locus within the above 150 kb genomic region, several PCR-based polymorphic markers, such as SSR, CAPS, dCAPS and AFLP were developed by the comparison of genomic DNA sequences between japonica obtained from RGP (Rice Genome Research Program) and indica in NCBI BLAST to perform PCR.

Among 1,165 lines of F₂ mapping population, which genotypes were verified by F₃ phenotyping from each F₂ progeny, 40 recombinants including sgr allele were initially isolated using two SSR markers, RM3636 and RM1553. Finally, two recombinants including sgr allele were identified between CAPS (33.9 kb) and SSR (38.2 kb) markers in AP005314, thus indicating that the sgr mutaton site is located within 4.3 kb in AP005314 DNA base sequence of the wild type japonica rice (Nipponbare) (FIG. 10B).

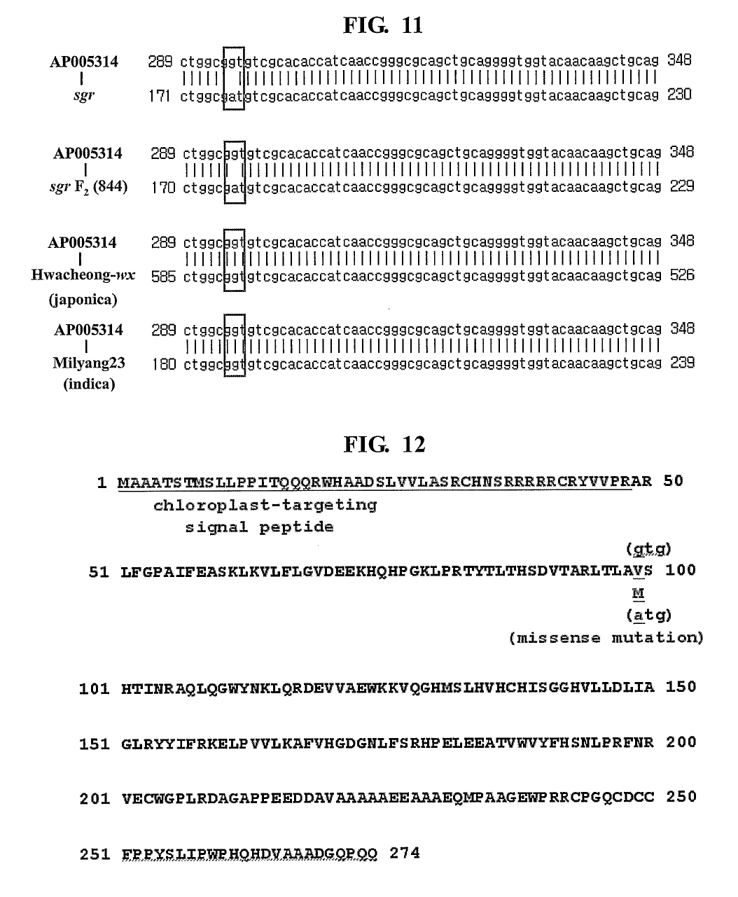

The putative 4.3 kb sgr mutation regions from the genomic DNAs of the sgr mutant generated from glutinous Hwacheong-wx rice by MNU mutation, the parental cultivar (japonica), the mapping parent Milyang23 (indica) and several sgr-genotype F₂ lines were amplified by PCR respectively, and sequenced them by cloning into pGemT vector (Promega, USA), followed by comparing based on the base sequence within 33.9˜38.2 kb among AP005314 base sequences which is a PAC clone of the wild type japonica rice Nipponbare genomic DNA (FIG. 11).

As a result, single base mutation, substituting A (adenine) for the 295^{th} base G (guanine) in ORF (open reading frame) among full-length cDNA base sequence registered as GenBank Accession NO: AK105810, which is registered as an unknown expressed gene whose function is not identified and exists in 4.3 kb region, was observed in all of the DNAs of the sgr mutants, thereby confirming the inducement of missense mutation by a substitution of methionine (atg) for the protein sequence 99^{th} amono acid valine (gtg) encoding by single base mutation of AK105810 gene in the sgr mutant (FIG. 12). Therefore, it is confirmed that AK105810 gene having normal base sequence (G) and amino acid (valine) is a SGR gene, and it participates in chlorophyll catabolism during senescence to encode SGR protein which induces chlorophyll degradation and consequently leaf yellowing.

And also, the present inventors registered the DNA sequence (SEQ ID NO: 1) of SGR gene derived from japonica Hwacheong glutinous rice along with amino acid sequence (SEQ ID NO: 30) of SGR protein in NCBI GenBank as a rice stay-green gene (OsSGR) (GenBank Accession NO:AY850134).

### EXAMPLE 8

### The Trait of SGR Gene Essential in Chlorophyll Catabolism

The SGR gene with SEQ ID NO: 1 encodes a polypeptide with SEQ ID NO: 30 which comprises 274 amino acids and molecular mass is 30,880 Da. The N terminus of the deduced SGR protein has a chloroplast-targeting signal peptide comprising 48 amino acids, and the result of the investigation by NCBI protein Domain Database confirmed the protein is a novel protein that has not been researched and reported until now (FIG. 12).

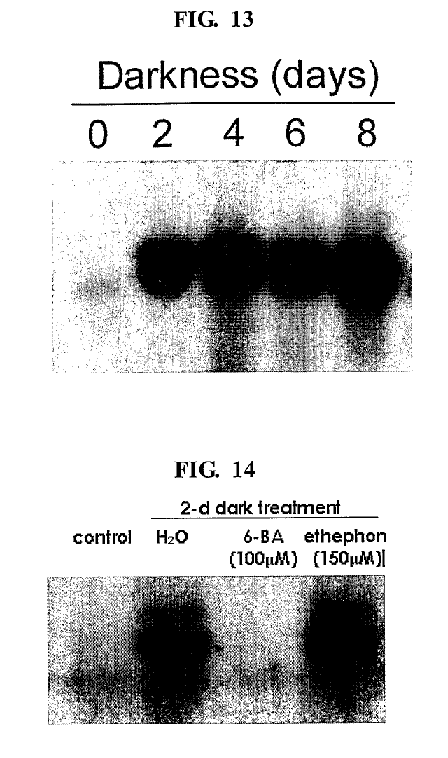

Meanwhile, in the case of senescence of wild type leaves were induced by dark treatment, as described in the FIG. 13, the SGR genes were highly expressed at 2^{nd} day after dark treatment (2 DAD), and the SGR mRNA level maintained high even when the senescing leaves turn yellow completely at 8 DAD. Also, as described in the FIG. 14, in the case of suppressing senescence by treating 6-benzylaminopurine (6-BA) which is a precursor of cytokinin, a senescence-suppressing hormone in plants, the expression of the SGR gene was suppressed even if senescence of the detached leaves of the wild type was induced highly at 2 DAD.

It confirmed that the SGR gene is a new unidentified senescence-associated gene (SAG) which induces leaf yellowing by participating essentially in the early stage of chlorophyll catabolism.

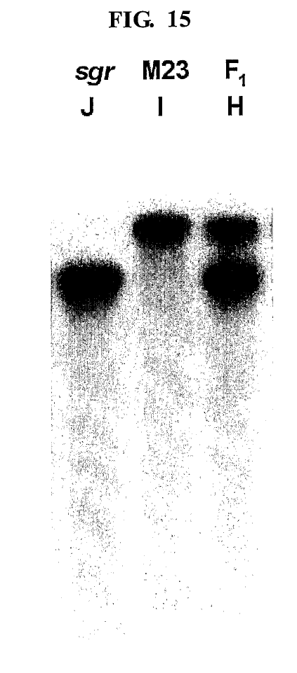

FIG. 15 shows the result of a southern blotting using the wild type SGR gene as a probe after treating, the genome DNA of japonica type stay-green mutant used in preparing genetic map (1 lane), indica type Milyang23 (2 lane) and F₁ hybrid plant obtained by hybridizing of them (3 lane), with Dra I. As described in the FIG. 15, SGR gene exists as a single copy in rice genome.

From the above results, adenine (A) substituted for the 295^{th} base guanine (G) of the SGR gene in the stay-green mutant gene as represented in SEQ ID NO: 3. Also, methionine (atg) substituted for the 99^{th} amino acid valine (gtg) among 274 amino acid sequences of the wild type SGR protein, in the protein encoded with sgr mutant gene as represented in SEQ ID NO: 4.

### EXAMPLE 9

### Analysis of Homology and Conserved Domain of SGR Protein in Other Plant

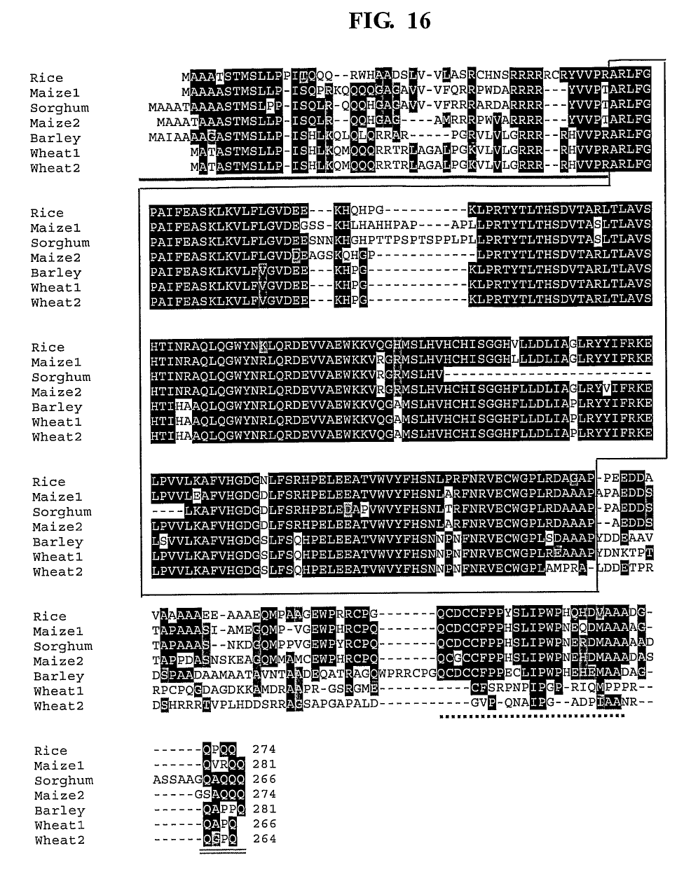

High homologous cDNA base sequences in other plants with DNA sequence of the SEQ ID NO: 1 according to the present invention by nucleotide blastn of NCBI GenBank (http://www.ncbi.nlm.nih.gov/BLAST), and Blast program of PlantGDB (http://www.plantgdb.org/cgi-bin/PlantGDBbfast) and TGI sequence search (http://tigrblast.tigr.org/tgi/) were identified to research the SGR domain region (conserved amino acid sequence region) by comparing SEQ ID NO: 30 polypeptide with amono acid sequence encoded with each cDNA sequence (FIG. 16 to FIG. 22).

In monocot among angiosperm, genes having high homology with SGR gene of rice (OsSGR) were found 1 (HvSGR) in Barley, 2 (TaSGR1 and TaSGR2) in Wheat, 2 (ZmSGR1 and ZmSGR2) in Maize. As represented in FIG. 16, polypeptide encoded with said genes has chloroplast-targeting signal peptide (solid line) of 1 to 48 region among SEQ ID NO: 30 amino acid in common, conserved amino acid sequence SGR domain I (box) with over 80% homology within 49 to 213 region, conserved amino acid sequence, SGR domain II (dotted line) within 246 to 267 region of the SEQ ID NO: 30 and SGR domain III (double solid line) with 2 to 3 conserved glutamine (Q) in C-terminus. However, SGR protein of the wheat was deficient in SGR domain II. In FIG. 16, chloroplast-targeting signal peptide amino acid sequence, identical amino acid and similar amino acid, are represented by solid line, black shadebox and gray shadebox, respectively. SGR domain I, SGR domain II and SGR domain III are represented by box, dotted line and double solid line, respectively.

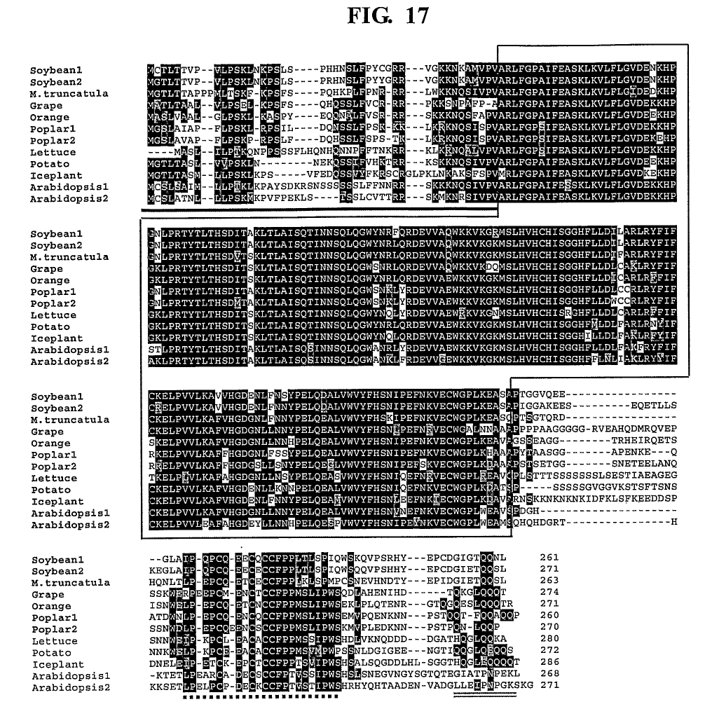

In dicot among angiosperm, genes having high homology with the SGR gene of rice (OsSGR; SEQ ID NO: 1) were found 2 ((GmSGR1; SEQ ID NO: 8) and (GmSGR2; SEQ ID NO: 9)) in Soybean, 1 (MtSGR; SEQ ID NO: 14) in Medicago truncatula, 1 (VvSGR; SEQ ID NO: 10) in Grape, 1 (CsSGR; SEQ ID NO: 13) in Orange, 2 ((PoSGR1; SEQ ID NO: 16) and (PoSGR2; SEQ ID NO: 17)) in Poplar, 1 (LsSGR; SEQ ID NO: 11) in Lettuce, 1 (StSGR; SEQ ID NO: 15) in Potato, 1 (McSGR; SEQ ID NO: 18) in Common Iceplant which is a composite, and 2 ((AtSGR1(At4g22920); SEQ ID NO: 19) and (AtSGR2(At4g11910); SEQ ID NO: 20)) in Arabidopsis thaliana. As represented in FIG. 17, the above SGR protein sequences of dicot have also chloroplast-targeting signal peptide in N-terminus region in common as in SGR protein sequences in monocot, SGR domain I with over 65% homology, SGR domain II and SGR domain III with 2 to 6 conserved glutamine (Q) in C-terminus. However, 2 SGR proteins of the Arabidopsis were deficient in SGR domain III. In FIG. 17, chloroplast-targeting signal peptide amino acid sequence, identical amino acid and similar amino acid are represented by solid line, black shadebox and gray shadebox, respectively. SGR domain I, SGR domain II and SGR domain III are represented by box, dotted line and double solid line, respectively.

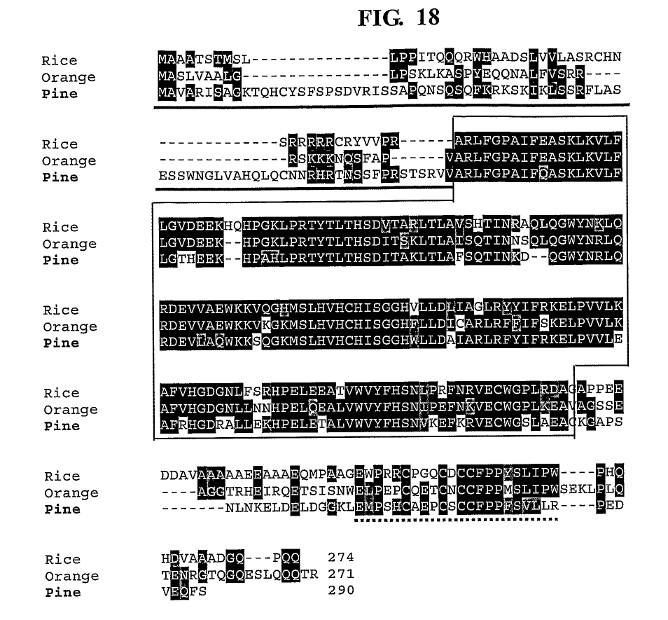

As a result of comparing 1 SGR protein sequence (PtSGR; SEQ ID NO: 12) found in pine which is gymnosperm with Rice OsSGR and Orange CsSGR protein sequences, they had chloroplast-targeting signal peptide, SGR domain I and SGR comain II and were deficient in SGR domain III (FIG. 18). In FIG. 18, chloroplast-targeting signal peptide amino acid sequence, identical amino acid and similar amino acid, are represented by solid line, black shadebox and gray shadebox, respectively. SGR domain I and SGR domain II are represented by box and dotted line, respectively.

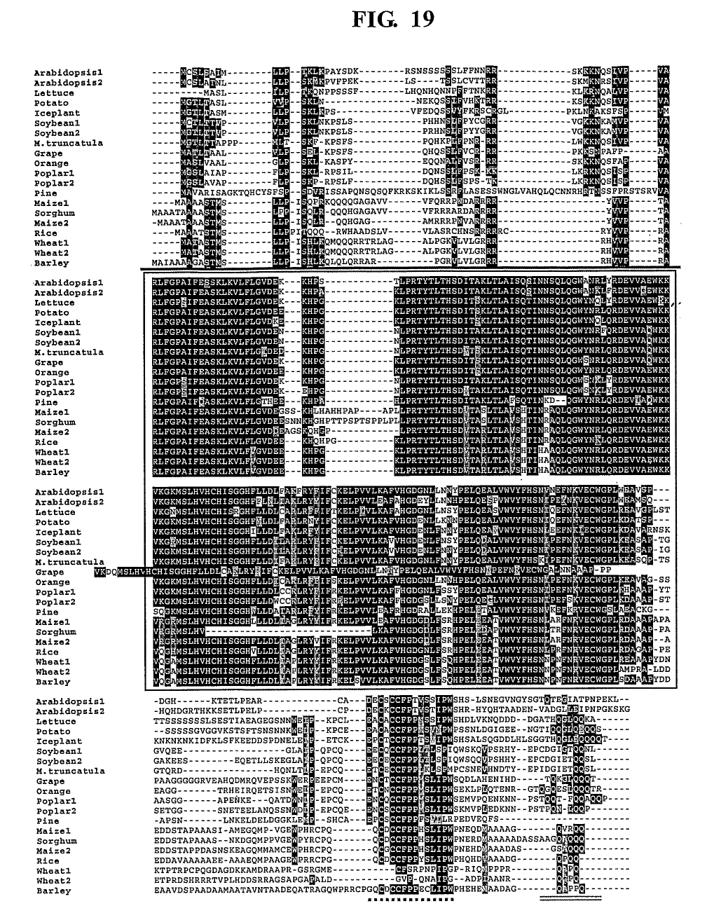

As a result of comparing amino acid sequences of SGR proteins in all plants examined in FIG. 16-18, they have chloroplast-targeting signal peptide (solid line) in N-terminus in common and high homologous SGR domain I (box). Except for TaSGR1 and TaSGR2 of wheat, they had SGR domain II (dotted line). In addition, except for SGRs of Arabidopsis and Pine, they had SGR domain III which contains 2 to 6 conserved glutamine (Q) in C-terminal region (FIG. 19). In FIG. 19, chloroplast-targeting signal peptide amino acid sequence, identical amino acid and similar amino acid are represented by solid line, black shadebox and gray shadebox, respectively. SGR domain I, SGR domain II and SGR domain III are represented by box, dotted line and double solid line, respectively.

Besides the above plants, 1 (SbSGR; SEQ ID NO: 7) gene encoding the amino acid sequence which is high homologous with SEQ ID NO: 30 was found in Sorghum and 1 (LeSGR; AY850152) in tomato, 1 (BvSGR; AY850153) in Sugarbeet, 1 (ZjSGR; AY850154) in Zoysia, 2 ((LjSGR1; AY850155) and (LjSGR2; AY850156)) in Lotus japonicus, 1 (SoSGR; AY850157) in Sugar cane, 1 (PiSGR; AY850158) in Spruce and 2 ((BnSGR1; AY850159) and (BnSGR2; AY850160)) in Rape were found, which are SGR genes showing only partial SGR cDNA sequence and amino acid sequence, due to the partial cDNA sequence information. The present inventors analyzed the base sequence of the each gene by RT-PCR, or searched from web database to register in NCBI GenBank.

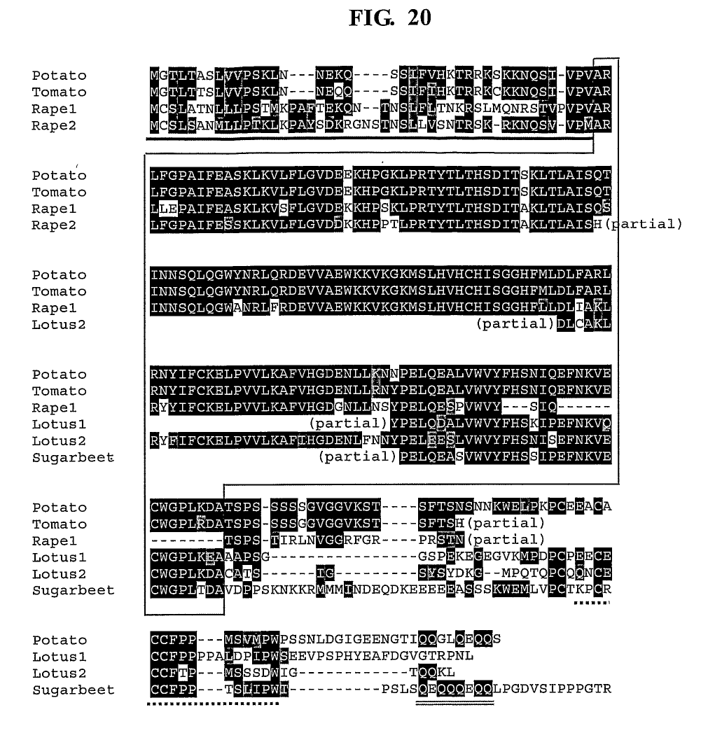

FIG. 20 is a boxshade form which represents the comparison between the partial sequence of protein of LsSGR (SEQ ID NO: 50) of Tomato, BvSGR (SEQ ID NO: 51) of Sugarbeet, LjSGR1 (SEQ ID NO: 53) and LjSGR2 (SEQ ID NO: 54) of Lotus japonicus, BnSGR1 (SEQ ID NO: 57) and BnSGR2 (SEQ ID NO: 58) of Rape which have high homology with SGR protein, among genes of dicotyledon and StSGR polypeptide of Potato (SEQ ID NO: 44). Chloroplast-targeting signal peptide, SGR domain I, SGR domain II and SGR domain III in C-terminal were marked with solid line, box, dotted line and double solid line, respectively in order to recognize the relative position of the partial sequences with ease. As described in FIG. 20, the partial sequence of LsSGR (SEQ ID NO: 50) of tomato and BnSGR1 (SEQ ID NO: 57) and BnSGR2 (SEQ ID NO: 58) of rape showed high homology with chloroplast-targeting signal peptide and SGR domain I regions of SGR protein. The partial sequence of BvSGR (SEQ ID NO: 51) of Sugarbeet and LjSGR2 (SEQ ID NO: 54) of Lotus japonicus showed high homology with partial SGR domain I, SGR domain II and SGR domain III of the SGR protein. LjSGR1 (SEQ ID NO: 53) showed high homology with partial SGR domain I and SGR domain II, however, it was deficient in SGR domain III as in the SGR proteins of Arabidopsis (AtSGR 1, AtSGR2), Pine (PtSGR) and Spruce (PiSGR).

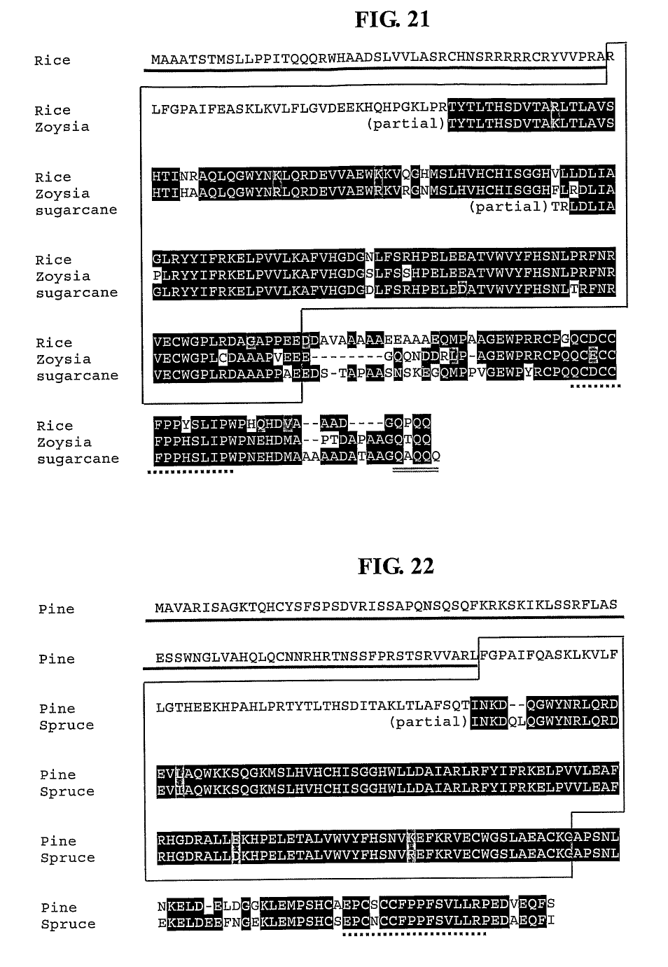

FIG. 21 is a boxshade form representing the comparison between the partial sequence of protein of ZjSGR (SEQ ID NO: 52) of Zoysia japonica and SoSGR (SEQ ID NO: 55) of Sugarcane which are high homologous with SGR protein, among genes of monocotyledon wherein partial DNA sequence is decoded, and SGR polypeptide of Rice (SEQ ID NO: 30), and FIG. 22 is a boxshade form representing the comparison between the partial sequence of protein of PiSGR (SEQ ID NO: 56) of Spruce which has high homology with SGR protein, among genes of gymnosperm and PtSGR polypeptide of Pine (SEQ ID NO: 41). All of these partial sequences have high homology with each SGR domain, thus making it possible to confirm that the proteins have the same function in spite of partial sequence information.

The 1˜2 copy(s) SGR genes encoding the SGR protein of said each plant exist in the each plant, and the genes have high homology (SGR domain I, II and III) with polypeptide of amino acid sequence (SEQ ID NO: 30), thereby supposing that they induce leaf yellowing by participating in chlorophyll catabolism as does SGR gene in rice.

Sequence information on the presentive SGR genes and SGR proteins which are derived from plant and whose function is confirmed, is shown in Table 2 below.

TABLE 2
SGR Gene and SGR Protein according to the present invention

| | | SEQ ID | SEQ ID | |
|---|---|---|---|---|
| | | NO: | NO: | |
| | | of SGR | of SGR | NCBI GenBank |
| Plants | Gene name | Gene | Protein | Accession NO: |
| Rice | OsSGR | 1 | 30 | AY850134 |
| Barley | HvSGR | 2 | 31 | AY850135 |
| Wheat | TaSGR1 | 3 | 32 | AY850136 |
| | TaSGR2 | 4 | 33 | AY850137 |
| Maize | ZmSGR1 | 5 | 34 | AY850138 |
| | ZmSGR2 | 6 | 35 | AY850139 |
| Sorghum | SbSGR | 7 | 36 | AY850140 |
| Soybean | GmSGR1 | 8 | 37 | AY850141 |
| | GmSGR2 | 9 | 38 | AY850142 |
| Grape | VvSGR | 10 | 39 | AY850143 |
| Lettuce | LsSGR | 11 | 40 | AY850144 |
| Pine | PtSGR | 12 | 41 | AY850145 |
| Orange | CsSGR | 13 | 42 | AY850146 |
| M. truncatula | MtSGR | 14 | 43 | AY850147 |
| Potato | StSGR | 15 | 44 | AY850148 |
| Poplar | PoSGR1 | 16 | 45 | AY850149 |
| | PoSGR2 | 17 | 46 | AY850150 |
| Iceplant | McSGR | 18 | 47 | AY850151 |
| Arabidopsis | AtSGR1 | 19 | 48 | AY850161 |
| | AtSGR2 | 20 | 49 | AY699948 |
| Tomato | LeSGR | 21 | 50 | AY850152 |
| Sugarbeet | BvSGR | 22 | 51 | AY850153 |
| Zoysia | ZjSGR | 23 | 52 | AY850154 |
| Lotus | LjSGR1 | 24 | 53 | AY850155 |
| | LjSGR2 | 25 | 54 | AY850156 |
| Sugar cane | SoSGR | 26 | 55 | AY850157 |
| Spruce | PiSGR | 27 | 56 | AY850158 |
| Rape | BnSGR1 | 28 | 57 | AY850159 |
| | BnSGR2 | 29 | 58 | AY850160 |

To confirm that the SGR genes of said plants participate in chlorophyll catabolism to induce leaf yellowing as does the SGR gene in rice, the expressions of AtSGR1 (At4g22920) and AtSGR2 (At4g11910) genes were examined during dark-induced senescence on the leaves of model plant, Arabidopsis thaliana. Specific primers from the base sequence of At4g22920 and At4g11910 genes were prepared, and then the RNA obtained from 0 (control), 1, 3, 6 and 9-day dark-treated mature leaf of Arabidopsis was amplified by RT-PCR using the primers.

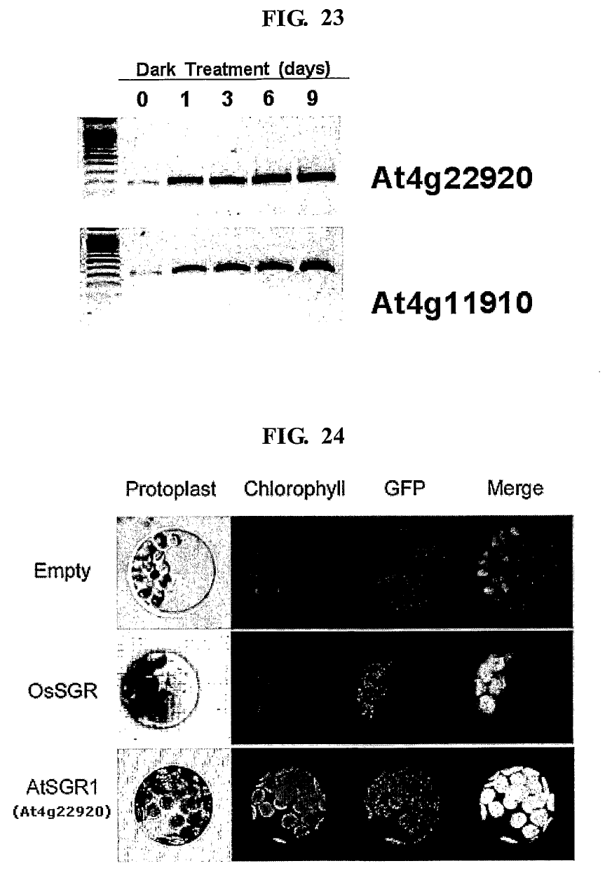

As a result, the above 2 genes were highly expressed by dark-induced senescence as represented in FIG. 23, thus making it possible to confirm that the At4g2290 and At4g11910 genes, as SGR gene in rice, are yellowing inducing genes which participates in chlorophyll catabolism during plant senescence.

All SGR proteins have the chlorophyll-targeting signal peptide information in N-terminal region in common. In order to confirm the fact, transient expression assay was performed with protoplast of the leaves of Arabidopsis. After constructing a vector by recombining OsSGR gene of rice having stop codon removed and cDNA sequences of AtSGR1, At4g22920 gene in front of GFP gene by CaMV35S promoter of pCAMBIA vector (CAMBIA, Australia), the constructed vector was transformed by treatment of PEG, the protoplast of the leaves of transformed Arabidopsis was observed with a fluorescence microscope (FIG. 24). As a result, it is confirmed that all GFP signals exist in chloroplast as described in FIG. 24, thereby proving that SGR proteins are accumulated in chloroplast after they are synthesized in ER.

### EXAMPLE 10

### Production of Stay-Green Mutant Arabidopsis by RNAi (RNA Interference) Gene Silencing

In order to prove the possibility of production of the stay-green mutant when the expression of SGR gene is inhibited at RNA level, or expressed SGR mRNA is degraded rapidly in cytoplasm or nucleus before being encoded to SGR protein by RNAi gene silencing mechanism, a stay-green mutant plant was produced by using the base sequence information of the SGR genes derived from various plants found in Example 9 and transforming into Arabidopsis.

After amplifying the most homologous SGR domain I obtained by comparing-ORF cDNA base sequences of Arabidopsis AtSGR1 (At4g22920) and AtSGR2 (At4g11910) using At4g11910 cDNA as a template, the PCR product was recombined to be controlled by CaMV35S promoter of pB7GWIWG2(II) (Flanders Interuniversity Institute for Biotechnology, Belgium) which is T-DNA vector for RNAi producing hairpin double-stranded RNA as Gateway vector, and then transformed into Columbia-0 (Col-0) ecotype which is the wild type Arabidopsis using floral dip method to collect T1 seeds, followed by obtaining 14 of T1 transgenic plants by the selection of Bastar Herbicides.

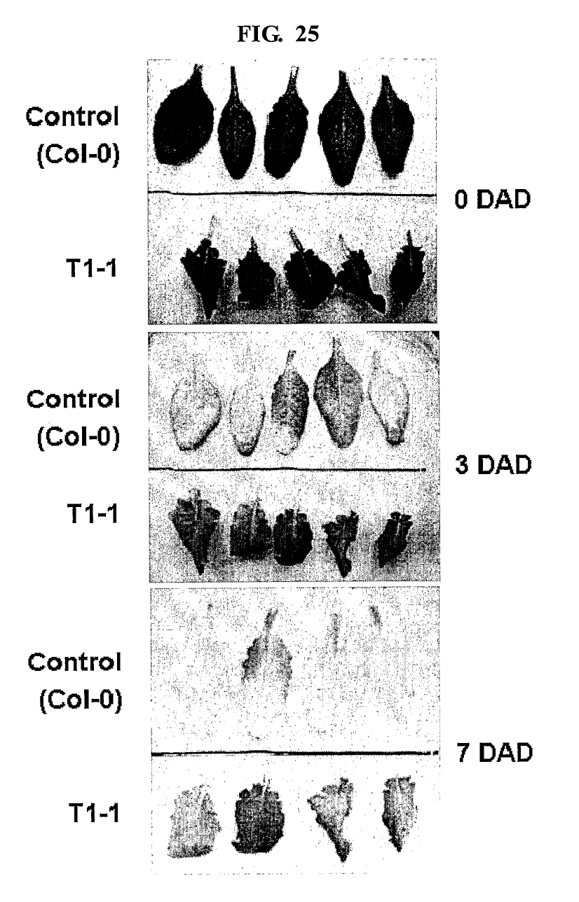

As a result of observation of the change from green to yellow with the passage of time by dark treatment at room temperature on mature leaves of T1-1 transgenic plant among 14 T1 transgenic plants and the control (Col-0), the leaf greenness of T1-1 lasted much longer than that of the control (Col-0) (FIG. 25). As described in FIG. 25, the leaves of T1-1 remained green while all leaves of control started to show leaf yellowing at 3 day after dark treatment (3DAD), and most of the control leaves turned yellow at 7 DAD.

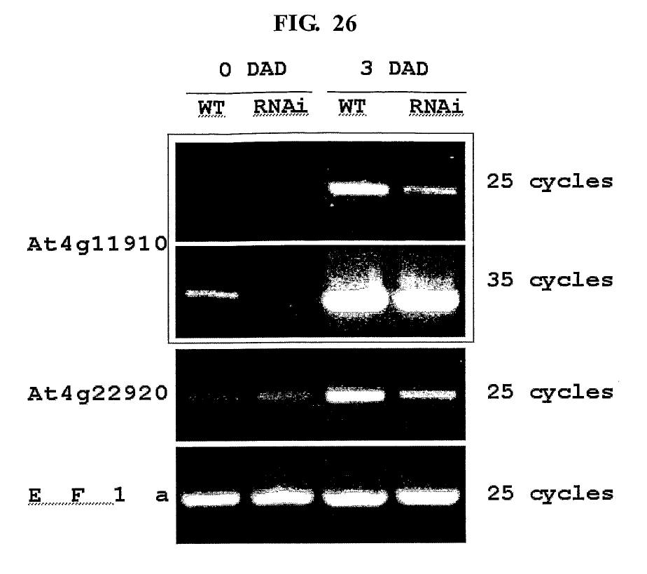

RNA levels of Arabidopsis SGR genes, At4g11910 and At4g22920, were compared between the wild type and the T1-1 transgenic plant by extracting total RNA from 3-DAD leaves of FIG. 25 (FIG. 26). As described in FIG. 26, the expression level of each gene was considerably reduced in T1-1 leaves by RNAi gene silencing transformation, thereby confirming that if SGR gene expression level is diminished, leaf greenness and freshness of leaves remains much longer.

Meanwhile, if the CaMV35S constitutive promoter is replaced with a promoter of SGR gene itself, or the known senescence-enhanced promoters which would be expected to induce much higher expression of SGR gene, specifically during senescence, the leaf greenness of RNAi transgenic plant during senescence can be lasted for much longer time by reducing the expression level of SGR gene.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

### INDUSTRIAL APPLICABILITY

As fully described above, the present invention provides a SGR gene participating in chlorophyll catabolism during plant senescence, thereby causing yellowing, a method for producing stay-green transgenic plants, which being characterized by mutating the SGR gene, suppressing the expression of the SGR gene, or deactivating the SGR protein which is encoded by the SGR gene, and stay-green mutant plants produced by said method.

According to the present invention, leaf greenness can be maintained for a long time by mutating the SGR gene, suppressing the expression of the SGR gene, or inactivating the SGR protein encoded by the SGR gene, thereby preventing leaf yellowing of plant in yellowing plant during plant senescence. Therefore, while storing of leaf vegetables and forage for feed-stuff after harvesting, the leaf freshness can be maintained much longer by long lasting greenness and higher concentration of protein, and the taste of rice is enhanced by reducing the concentration of seed protein resulted from low efficiency of nitrogen remobilization from leaf to seed during seed grain filling in cereal grain, the cost of producing high quality beer may be reduced in the case of malt barley grains produced by stay-green barley. Especially, turfgrasses in golf courses or parks can be remained green for a long time even in midsummer drought or midwinter, thereby being expected to be useful for tourism, landscape architecture and leisure industries.

1. A SGR gene encoding a polypeptide comprising amino acid sequence having at least 60% homology with SGR domain I which is conserved amino acid sequence region of 49˜207 among amino acid sequence of SEQ ID NO: 30, and inducing leaf yellowing by participating in chlorophyll catabolism during plant senescence.

2. The SGR gene according to claim 1, wherein the polypeptide comprises a chloroplast-targeting signal peptide sequence, SGR domain II, and/or SGR domain III which contains 2˜6 conserved glutamines (Qs) in C-terminal region.

3. The SGR gene according to claim 1, wherein the polypeptide comprises the amino acid sequence selected from the group consisting of SEQ ID NOs: 30 to 50 and 57.

4. The SGR gene according to claim 3, wherein the SGR gene comprises the DNA sequence selected from the group consisting of SEQ ID NOs:1 to 21 and 28.

5. A polypeptide encoded by the SGR gene of claim 4.

6. A recombinant vector comprising the SGR gene of claim 1.

7. A microorganism transformed with the recombinant vector of claim 6.

8. A plant transformed with the SGR gene of claim 1 or claim 2.

9. A method for producing a stay-green mutant plant, which comprises mutating SGR gene of yellowing plants or fragments thereof.

10. The method according to claim 9, wherein the SGR gene encodes the polypeptide comprising amino acid sequence having at least 60% homology with SGR domain I which is conserved amino acid sequence region of 49˜207 among amino acid sequence of SEQ ID NO: 30.

11. The method according to claim 10, wherein the polypeptide comprises the chloroplast-targeting signal peptide sequence and SGR domain II, and/or SGR domain III which contains 2˜6 conserved glutamines (Qs) in C-terminal region.

12. The method according to claim 10, wherein the SGR gene comprises the base sequence selected from the group consisting of SEQ ID NOs:1 to 21 and 28.

13. The method according to claim 9, wherein the SGR gene fragment comprises the DNA sequence selected from the group consisting of SEQ ID NOs: 21 to 29.

14. The method according to claim 9, wherein the mutating of SGR gene is carried out by deleting a part of base of said gene, substituting other singular or plural bases for a part of base of said gene, or adding other singular or plural bases to said gene.

15. The method according to claim 12, wherein A substitutes for the 295^{th} base G in the SGR gene of SEQ ID NO: 1.

16. A stay-green mutant plant produced by the method of claim 9.

17. A method for producing a stay-green mutant plant, which comprises suppressing the expression of the SGR gene in yellowing plant.

18. The method according to claim 17, wherein the SGR gene encodes the polypeptide comprising amino acid sequence having at least 60% homology with SGR domain I which is conserved amino acid sequence region of 49˜207 among amino acid sequence of SEQ ID NO: 30.

19. The method according to claim 18, wherein the polypeptide comprises the chloroplast-targeting signal peptide sequence and SGR domain II, and/or SGR domain III which contains 2˜6 conserved glutamines (Qs) in C-terminal region.

20. The method according to claim 18, wherein the SGR gene comprises the base sequence selected from the group consisting of SEQ ID NOs:1 to 21 and 28.

21. The method according to claim 17, wherein suppressing the expression of the SGR gene is performed by gene silencing technique.

22. A stay-green mutant plant produced by the method of claim 17.

23. A method for producing a stay-green mutant plant, which comprises the steps of:
(a) obtaining a recombinant vector by introducing a SGR gene or a fragment thereof originated from target plant to be mutated, to T-DNA vector; and
(b) transforming a wild type plant with the recombinant vector.

24. The method according to claim 23, wherein the SGR gene encodes the polypeptide comprising amino acid sequence having at least 60% homology with SGR domain I which is conserved amino acid sequence region of 49˜207 among amino acid sequence of SEQ ID NO: 30.

25. The method according to claim 24, wherein the polypeptide comprises the chloroplast-targeting signal peptide sequence and SGR domain II, and/or SGR domain III which contains 2˜6 conserved glutamines (Qs) in C-terminal region.

26. The method according to claim 24, wherein the SGR gene comprises the base sequence selected from the group consisting of SEQ ID NOs:1 to 21 and 28.

27. The method according to claim 23, wherein the SGR gene fragment comprises the DNA sequence selected from the group consisting of SEQ ID NOs: 21 to 29.

28. The method according to claim 23, wherein the T-DNA vector is a vector for RNAi which induces gene silencing by making the double-stranded RNA (dsRNA) in a transgenic plant.

29. The method according to claim 23, wherein the recombinant vector comprises CaMV35s promoter or senescence-enhanced promoter.

30. A stay-green mutant plant produced by the method of claim 23.

31. A method for producing a stay-green mutant plant, which comprises inactivating the protein encoded by the SGR gene in yellowing plant.

32. The method according to claim 31, wherein the SGR gene encodes the polypeptide comprising amino acid sequence having at least 60% homology with SGR domain I which is conserved amino acid sequence region of 49˜207 among amino acid sequence of SEQ ID NO: 30.

33. The method according to claim 32, wherein the polypeptide comprises the chloroplast-targeting signal peptide sequence and SGR domain II, and/or SGR domain III which contains 2˜6 conserved glutamines (Qs) in C-terminal region.

34. The method according to claim 32, wherein the SGR gene comprises the base sequence selected from the group consisting of SEQ ID NOs: 1 to 21 and 28.

35. A stay-green mutant plant produced by the method of claim 31.
